(19) United States
(12) Patent Application Publication
(10) Pub. No.: US 2021/0062017 A1
(43) Pub. Date: Mar. 4, 2021

(54) BRANCHED ACETAL COALESCING AIDS

(71) Applicant: Eastman Chemical Company, Kingsport, TN (US)

(72) Inventors: Matthew Allen Boone, Kingsport, TN (US); Stephanie Kay Clendennen, Kingsport, TN (US)

(73) Assignee: Eastman Chemical Company, Kingsport, TN (US)

(21) Appl. No.: 16/560,146

(22) Filed: Sep. 4, 2019

(51) Int. Cl. C09D 7/63; C07C 43/303; C08K 5/06

(52) U.S. Cl. CPC C09D 7/63; C08K 5/06; C07C 43/303

(57) ABSTRACT

The present application relates to branched acetal compounds. The branched acetal compounds have utility as additives in paint and coating compositions. The branched acetal compounds exhibit reduced Volatile Organic Content (VOC). When added to a paint or coating composition, the branched acetal compositions of the present application provide satisfactory coating coalescing activity and reduced VOC content.

### FIELD OF THE INVENTION

The application relates to chemistry generally. This application also relates to branched acetal coalescents and coating compositions made from the coalescents.

### BACKGROUND OF THE INVENTION

Coalescing aids are added to water-based paints and act as a temporary plasticizer in latex emulsions. The coalescing aid lowers the glass transition temperature (Tg) of the latex polymer. As the paint dries the polymers that have been softened by the coalescing aid are allowed to flow together and form a film after the water has left the system. Coalescing aids that are volatile evaporate out of the film. This allows the polymer to return to the original Tg therefore giving harder films for better block and print resistant coatings.

New coalescents have been introduced to the coatings industry to address performance needs related to air quality regulations, film properties and consumer preferences. Non-volatile coalescing aids are increasingly used in latex paints. In particular, these materials offer reduced volatility and sometimes improved odor characteristics than the most commonly used latex coating coalescent, 2,2,4-trimethyl-1,3-pentanediol monoisobutyrate (Texanol™). Since they do not evaporate out of the coating, non-volatile coalescing aids function more like plasticizers. The main drawback of non-volatile coalescing aids is that they do not allow the polymer to return to its original Tg, and the latex polymers remain soft and tacky which can cause poor block and print resistance and poor weatherability.

There is a need for paint additives that facilitate the low temperature coalescence of latex particles to form a continuous film, even at application temperatures below the latex polymer T_{g}, while still resulting in a film without compromising hardness, block or print resistance, scrub resistance, weatherability or solvent resistance. In particular, a need exists for waterborne coating compositions which may be formulated as a single, shelf-stable composition but which exhibit efficient film formation imparting desired properties to the resulting coating.

Other beneficial features of a good coalescing aid include low water solubility, ease of addition to paint formulations, compatibility with multiple formulations, high coalescing efficiency, low freezing point, low foaming and good hydrolytic stability. A good coalescing aid will be compatible with most latex polymers, is easily added to formulations, has low volatility and odor, and provides good color development properties.

### SUMMARY

The present application discloses a compound of Formula I:

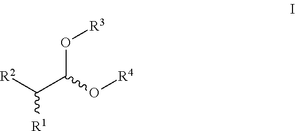

R¹ is hydrogen or (C₁₋₁₂)alkyl;
R² is (C₁₋₁₂)alkyl;
R³ is

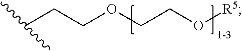

R⁴ is

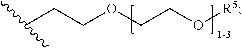

and
each R⁵ is (C₁₋₆)alkyl or (C₁₋₆)alkenyl,
wherein when R¹ is hydrogen, R⁴ is not ethyl, hexyl or decyl,
wherein when R¹ is methyl, R⁴ is not methyl.

The present application also discloses a composition comprising:
(1) a compound according to Formula I:

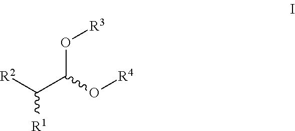

R¹ is hydrogen, or (C₁₋₁₂)alkyl;
R² is (C₁₋₁₂)alkyl;
R³ is

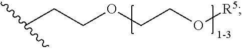

R⁴ is

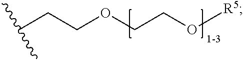

and
each R⁵ is (C₁₋₆)alkyl or (C₁₋₆)alkenyl; and
(2) a latex polymer;
wherein the compound of Formula I is present from about 1 to about 20 phr relative to the sum total of the latex polymer.

### DETAILED DESCRIPTION

### Definitions

In this specification and in the claims that follow, reference will be made to a number of terms, which shall be defined to have the following meanings.

“Alkyl” means an aliphatic hydrocarbon. The alkyl can specify the number of carbon atoms, for example (C₁₋₅)alkyl. Unless otherwise specified, the alkyl group can be unbranched or branched. In one embodiment, the alkyl group is branched. In one embodiment, the alkyl group is unbranched. Non-limiting examples of alkanes include methane, ethane, propane, isopropyl (i.e., branched propyl), butyl, and the like.

“Alkenyl” means an aliphatic hydrocarbon with one or more unsaturated carbon-carbon bonds. The alkenyl can specify the number of carbon atoms, for example (C₂₋₁₂)alkenyl. Unless otherwise specified, the alkyl group can be unbranched or branched. In one embodiment, the alkyl group is branched. In one embodiment, the alkyl group is unbranched. Non-limiting examples of alkanes include ethenyl, propenyl, butenyl, hexa-3,5-dienyl, and the like.

Values may be expressed as “about” or “approximately” a given number. Similarly, ranges may be expressed herein as from “about” one particular value and/or to “about” or another particular value. When such a range is expressed, another aspect includes from the one particular value and/or to the other particular value. Similarly, when values are expressed as approximations, by use of the antecedent “about,” it will be understood that the particular value forms another aspect.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the terms “having,” “has,” and “have” have the same open-ended meaning as “comprising,” “comprises,” and “comprise” provided above.

As used herein, the terms “including,” “includes,” and “include” have the same open-ended meaning as “comprising,” “comprises,” and “comprise” provided above.

“Chosen from” as used herein can be used with “or” or “and.” For example, Y is chosen from A, B, and C means Y can be individually A, B, or C. Alternatively, Y is chosen from A, B, or C means Y can be individually A, B, or C, or a combination of A and B, A and C, B and C, or A, B, and C.

### Composition of Matter

The present application discloses a compound of Formula I:

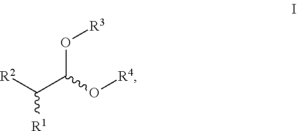

wherein: R¹ is hydrogen or (C₁₋₁₂)alkyl; R² is (C₁₋₁₂)alkyl; R³ is

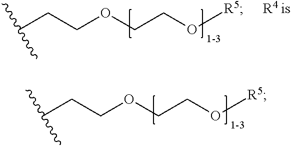

and each R⁵ is (C₁₋₆)alkyl or (C₁₋₆)alkenyl,
wherein when R¹ is hydrogen, R² is not ethyl, hexyl or decyl,
wherein when R¹ is methyl, R² is not methyl.

In one embodiment, R¹ is hydrogen. In one class of this embodiment, R² is methyl, propyl, butyl, pentyl, heptyl, octyl, or nonyl. In one subclass of this class, R² is methyl. In one subclass of this class, R² is propyl. In one subclass of this class, R² is butyl. In one subclass of this class, R² is pentyl. In one subclass of this class, R² is heptyl. In one subclass of this class, R² is octyl. In one subclass of this class, R² is nonyl.

In one embodiment, R¹ is (C₁₋₁₂)alkyl. In one embodiment, R¹ is methyl, ethyl, propyl, butyl, pentyl, hexyl, heptyl, octyl, nonyl, or decyl. In one class of this embodiment, R¹ is methyl. In one subclass of this class, R² is ethyl, propyl, butyl, pentyl, hexyl, heptyl, octyl, nonyl, or decyl. In one subclass of this class, R² is ethyl. In one subclass of this class, R² is propyl. In one subclass of this class, R² is butyl. In one subclass of this class, R² is pentyl. In one subclass of this class, R² is hexyl. In one subclass of this class, R² is heptyl. In one subclass of this class, R² is octyl. In one subclass of this class, R² is nonyl. In one subclass of this class, R² is decyl.

In one class of this embodiment, ethyl. In one subclass of this class, R² is methyl, ethyl, propyl, butyl, pentyl, hexyl, heptyl, octyl, nonyl, or decyl. In one class of this embodiment, R¹ is propyl. In one subclass of this class, R² is methyl, ethyl, propyl, butyl, pentyl, hexyl, heptyl, octyl, nonyl, or decyl. In one class of this embodiment, R¹ is butyl. In one subclass of this class, R² is methyl, ethyl, propyl, butyl, pentyl, hexyl, heptyl, octyl, nonyl, or decyl. In one class of this embodiment, R¹ is pentyl. In one subclass of this class, R² is methyl, ethyl, propyl, butyl, pentyl, hexyl, heptyl, octyl, nonyl, or decyl. In one class of this embodiment, R¹ is hexyl. In one subclass of this class, R² is methyl, ethyl, propyl, butyl, pentyl, hexyl, heptyl, octyl, nonyl, or decyl. In one class of this embodiment, R¹ is heptyl. In one subclass of this class, R² is methyl, ethyl, propyl, butyl, pentyl, hexyl, heptyl, octyl, nonyl, or decyl. In one class of this embodiment, R¹ is octyl. In one subclass of this class, R² is methyl, ethyl, propyl, butyl, pentyl, hexyl, heptyl, octyl, nonyl, or decyl. In one class of this embodiment, R¹ is nonyl. In one subclass of this class, R² is methyl, ethyl, propyl, butyl, pentyl, hexyl, heptyl, octyl, nonyl, or decyl. In one class of this embodiment, R¹ is decyl. In one subclass of this class, R² is methyl, ethyl, propyl, butyl, pentyl, hexyl, heptyl, octyl, nonyl, or decyl.

In one embodiment, R¹ is methyl or ethyl. In one embodiment, R¹ is ethyl, propyl, butyl, pentyl, hexyl, heptyl, octyl, nonyl, or decyl.

In one embodiment, R² is methyl, ethyl, propyl, butyl, pentyl, hexyl, heptyl, octyl, nonyl, or decyl. In one class of this embodiment, R² is methyl. In one class of this embodiment, R² is ethyl. In one class of this embodiment, R² is propyl. In one class of this embodiment, R² is butyl. In one class of this embodiment, R² is pentyl. In one class of this embodiment, R² is hexyl. In one class of this embodiment, R² is heptyl. In one class of this embodiment, R² is octyl. In one class of this embodiment, R² is nonyl. In one class of this embodiment, R² is decyl. In one embodiment, R² is propyl, butyl, octyl, or nonyl.

In one embodiment, R³ is

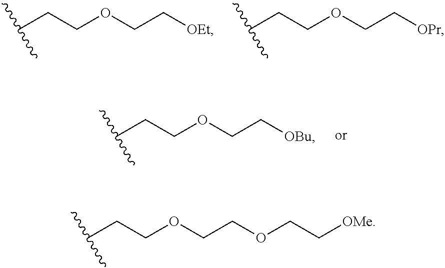

In one class of this embodiment, R⁴

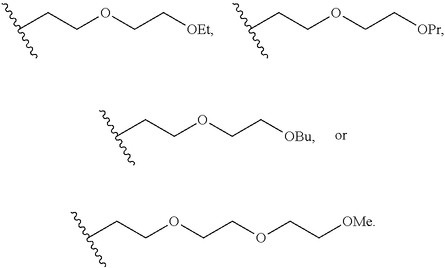

In one class of this embodiment, R⁴ is

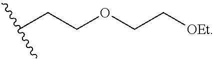 [SMILES: *CCOCCOCC]

In one class of this embodiment, R⁴ is

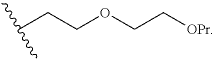

In one class of this embodiment, R⁴ is

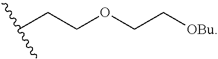

In one class of this embodiment, R⁴ is

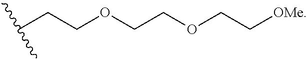 [SMILES: *CCOCCOCCOC]

In one embodiment, R³ is

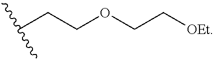 [SMILES: *CCOCCOCC]

In one class of this embodiment, R⁴ is

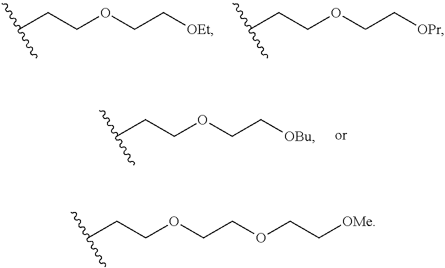

In one class of this embodiment, R⁴ is

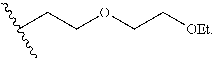 [SMILES: *CCOCCOCC]

In one class of this embodiment, R⁴ is

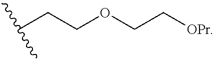

In one class of this embodiment, R⁴ is

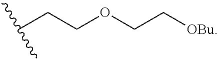

In one class of this embodiment, R⁴ is

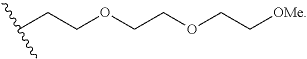 [SMILES: *CCOCCOCCOC]

In one embodiment, R³ is

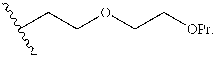

In one class of this embodiment, R⁴ is

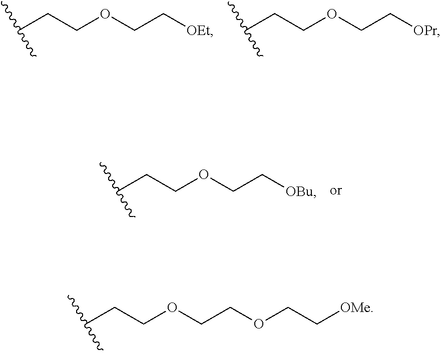

In one class of this embodiment, R⁴ is

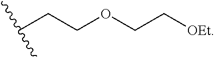 [SMILES: *CCOCCOCC]

In one class of this embodiment, R⁴ is

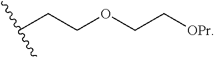

In one class of this embodiment, R⁴ is

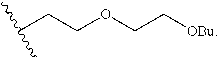

In one class of this embodiment, R⁴ is

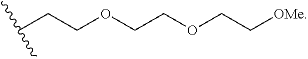 [SMILES: *CCOCCOCCOC]

In one embodiment, R³ is

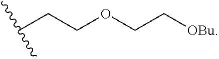

In one class of this embodiment, R⁴ is

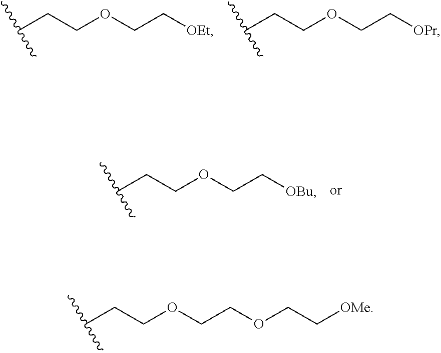

In one class of this embodiment, R⁴ is

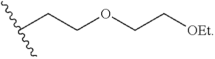 [SMILES: *CCOCCOCC]

In one class of this embodiment, R⁴ is

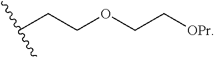

In one class of this embodiment, R⁴ is

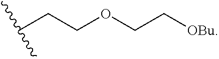

In one class of this embodiment, R⁴ is

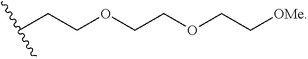 [SMILES: *CCOCCOCCOC]

In one embodiment, R³ is

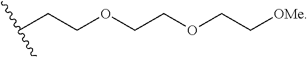 [SMILES: *CCOCCOCCOC]

In one class of this embodiment, R⁴ is

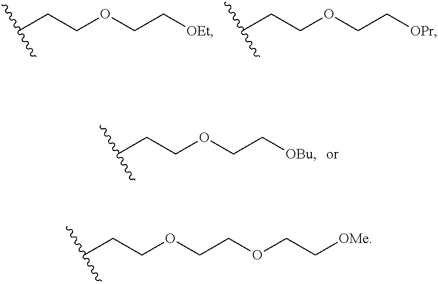

In one class of this embodiment, R⁴ is

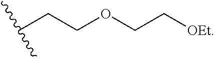 [SMILES: *CCOCCOCC]

In one class of this embodiment, R⁴ is

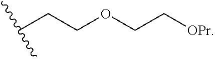

In one class of this embodiment, R⁴ is

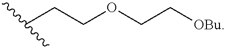

In one class of this embodiment, R⁴ is

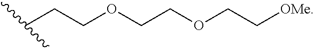 [SMILES: *CCOCCOCCOC]

In one embodiment, R⁴ is

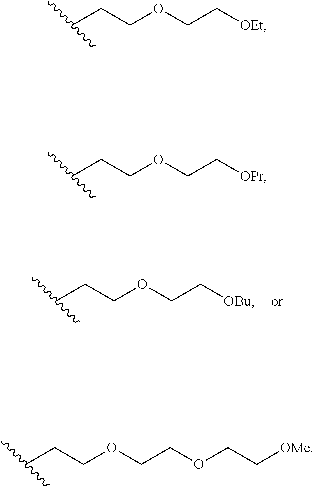

In one embodiment, the compound of Formula I is chosen from:

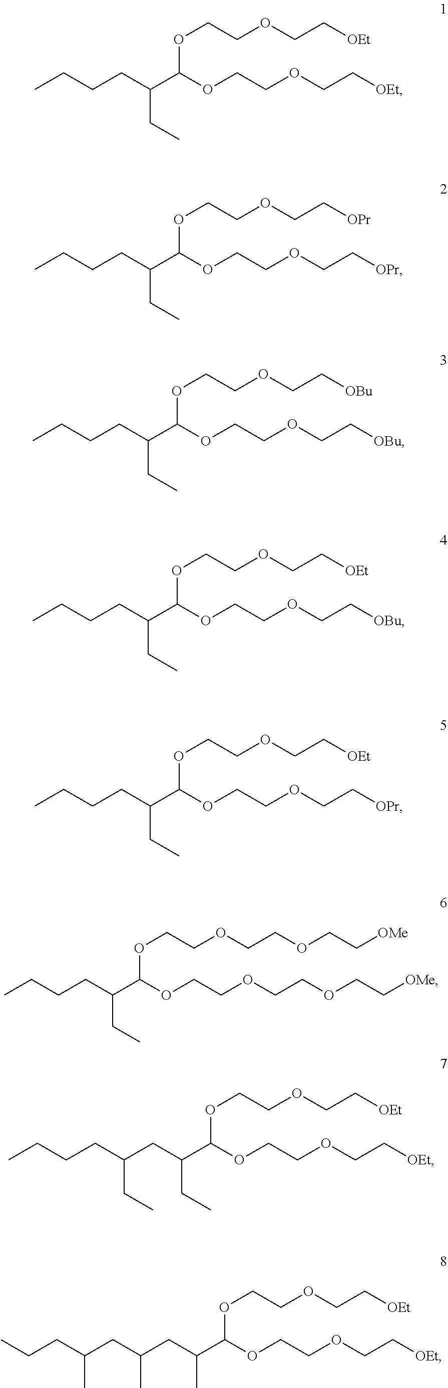

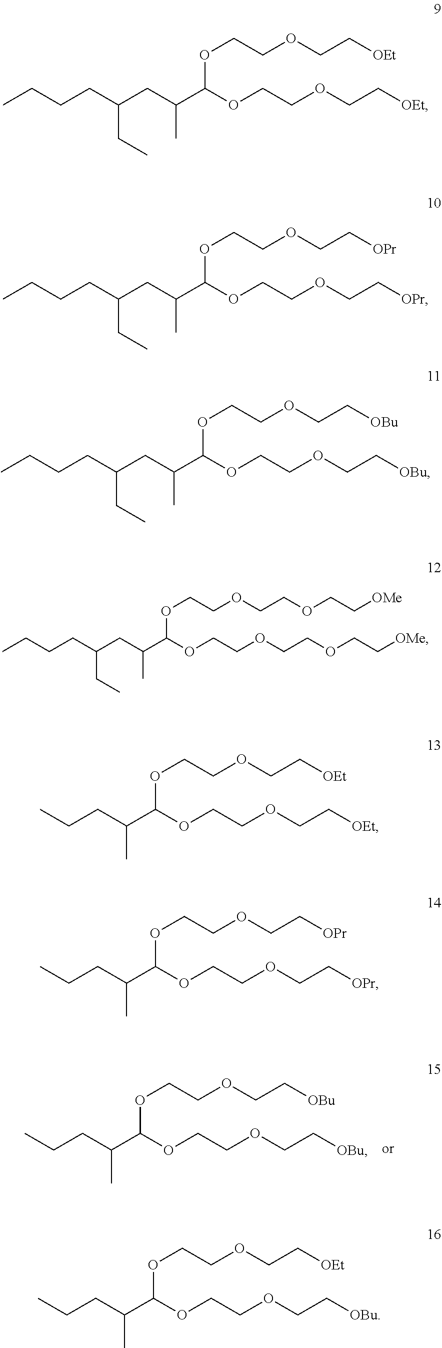

In one class of this embodiment, the compound of Formula I is

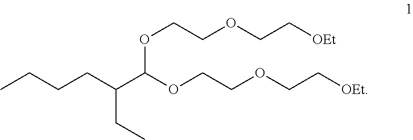 [SMILES: CCCCC(CC)C(OCCOCCOCC)OCCOCCOCC]

In one class of this embodiment, the compound of Formula I is

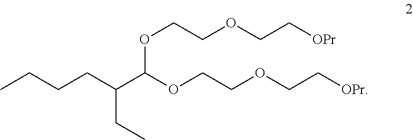

In one class of this embodiment, the compound of Formula I is

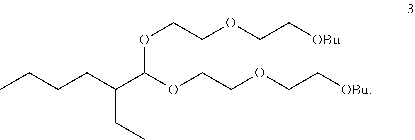

In one class of this embodiment, the compound of Formula I is

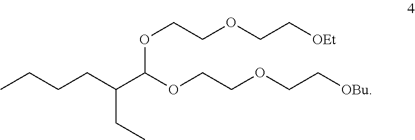

In one class of this embodiment, the compound of Formula I is

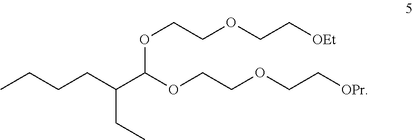

In one class of this embodiment, the compound of Formula I is

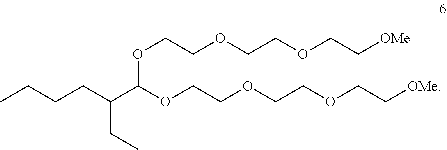 [SMILES: CCCCC(CC)C(OCCOCCOCCOC)OCCOCCOCCOC]

In one class of this embodiment, the compound of Formula I is

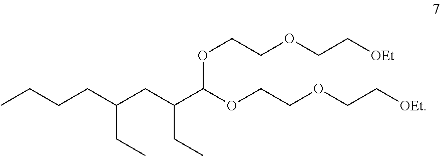 [SMILES: CCCCC(CC)CC(CC)C(OCCOCCOCC)OCCOCCOCC]

In one class of this embodiment, the compound of Formula I is

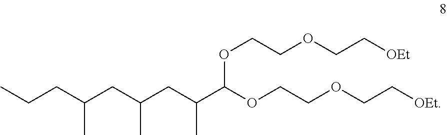 [SMILES: CCCC(C)CC(C)CC(C)C(OCCOCCOCC)OCCOCCOCC]

In one class of this embodiment, the compound of Formula I is

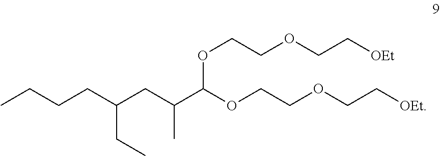 [SMILES: CCCCC(CC)CC(C)C(OCCOCCOCC)OCCOCCOCC]

In one class of this embodiment, the compound of Formula I is

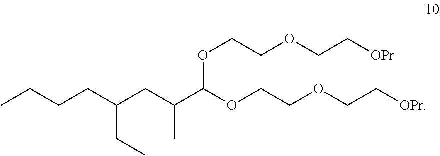

In one class of this embodiment, the compound of Formula I is

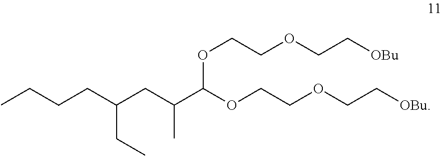

In one class of this embodiment, the compound of Formula I is

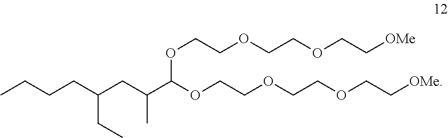 [SMILES: CCCCC(CC)CC(C)C(OCCOCCOCCOC)OCCOCCOCCOC]

In one class of this embodiment, the compound of Formula I is

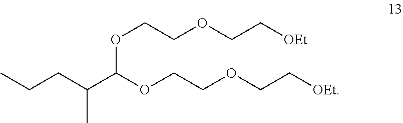 [SMILES: CCCC(C)C(OCCOCCOCC)OCCOCCOCC]

In one class of this embodiment, the compound of Formula I is

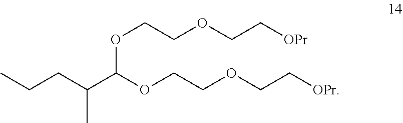

In one class of this embodiment, the compound of Formula I is

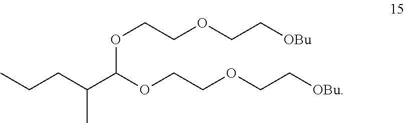

In one class of this embodiment, the compound of Formula I is

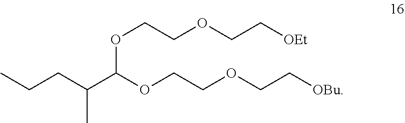

In one embodiment, the compound of Formula I has a volatile organic content of less than 50 wt % according to ASTM D6886. In one embodiment, the compound of Formula I has a volatile organic content of less than 40 wt % according to ASTM D6886. In one embodiment, the compound of Formula I has a volatile organic content of less than 30 wt % according to ASTM D6886. In one embodiment, the compound of Formula I has a volatile organic content of less than 20 wt % according to ASTM D6886. In one embodiment, the compound of Formula I has a volatile organic content of less than 10 wt % according to ASTM D6886. In one embodiment, the compound of Formula I has a volatile organic content of less than 5 wt % according to ASTM D6886.

### Composition

The compounds disclosed in the present application exhibit a low volatile organic content (less than 50 wt %, but as low as 0.7 wt % according to ASTM D6886) and formulate and have coalescing properties similarly or better than coalescing aids such as 2,24-trimethylpentane-1,3-diol monoisobutyrate. Therefore, the compounds disclosed in the present application are desirable in coating compositions.

The present application also discloses a composition comprising the compound of Formula I. In one embodiment, the composition further comprises a polymer. In one class of this embodiment, the polymer is a latex polymer.

The present application discloses a composition comprising the compound of Formula

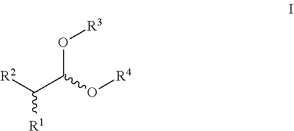

R¹ is hydrogen, or (C₁₋₁₂)alkyl; R² is (C₁₋₁₂)alkyl; R³ is

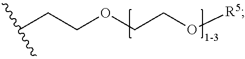

R⁴ is

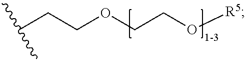

and each R⁵ is (C₁₋₆)alkyl or (C₁₋₆)alkenyl; and a polymer. In one class of this embodiment, the polymer is a latex polymer. In one subclass of this class, the latex polymer has a T_{g} in the range of from about −20° C. to about 100° C. In one subclass of this class, the latex polymer has a T_{g} in the range of from about 2° C. to about 60° C.

In one subclass of this class, the latex polymer is chosen from an acrylic, a vinyl acrylic, a styrene butadiene or a styrene acrylic latex polymer. In one sub-subclass of this subclass, the latex polymer is an acrylic latex polymer. In one sub-subclass of this subclass, the latex polymer is a vinyl latex polymer. In one sub-subclass of this subclass, the latex polymer is styrene butadiene latex polymer. In one sub-subclass of this subclass, the latex polymer is a styrene acrylic latex polymer.

In one class of this embodiment, the compound of Formula I is present from about 1 to about 20 phr relative to the sum total of the polymer. In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 5° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 2° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one class of this embodiment, the compound of Formula I is present from about 1 to about 15 phr relative to the sum total of the polymer. In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 5° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 2° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one class of this embodiment, the compound of Formula I is present from about 1 to about 10 phr relative to the sum total of the polymer. In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 5° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 2° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one class of this embodiment, the compound of Formula I is present from about 1 to about 8 phr relative to the sum total of the polymer. In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 5° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 2° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one class of this embodiment, the compound of Formula I, II or III is present from about 1 to about 6 phr relative to the sum total of the polymer. In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 5° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 2° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one class of this embodiment, the compound of Formula I, II or III is present from about 1 to about 5 phr relative to the sum total of the polymer. In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 5° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 2° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one class of this embodiment, the compound of Formula I, II or III is present from about 1 to about 4 phr relative to the sum total of the polymer. In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 5° C. In one sub-subclass of this sub class, the polymer is a latex polymer. In one sub-sub-subclass of this sub-subclass, the latex polymer has a Tg in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 60° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

In one subclass of this class, the composition has a minimum film formation temperature in the range of from about −35° C. to about 2° C. In one sub-subclass of this sub class, the polymer is a latex polymer. In one sub-sub-subclass of this sub-subclass, the latex polymer has a T_{g} in the range of from about −20° C. to about 100° C. In one sub-sub-subclass of this sub-subclass, the latex polymer has a T_{g} in the range of from about 2° C. to about 60° C. In one sub-sub-subclass of this sub-subclass, the latex polymer is chosen from an acrylic, a vinyl acrylic, a styrene butadiene or a styrene acrylic latex polymer.

The compounds (i.e., Formula I) of the present invention useful as coalescents according to the invention include those having a weight percent volatile content of less than 50%, as measured according to ASTM Method D6886. This test may be conducted generally by heating the sample in a forced air oven at 110° C. for 60 minutes. The weight loss after the test is deemed to result from a loss of volatiles originally present in the sample; the percent volatile present in the original sample may then be calculated. Although the cited test can be conducted on coating compositions containing other components such as latex polymers, the values cited herein may be obtained from a sample of the coalescent itself. The weight percent volatile of a coalescent may be used herein as a yardstick to measure the amount of VOC the coalescent would contribute to the VOC of a coating composition.

Examples of the “latex polymers” useful according to the invention include aqueous vinyl polymers, which are the reaction products of one or more ethylenically unsaturated monomers. Examples of the ethylenically unsaturated monomers include, but are not limited to, styrene, methyl acrylate, methyl methacrylate, ethyl acrylate, ethyl methacrylate, butyl acrylate, butyl methacrylate, isobutyl acrylate, isobutyl methacrylate, ethylhexyl acrylate, 2-ethylhexyl methacrylate, 2-ethylhexyl acrylate, isoprene, octyl acrylate, octyl methacrylate, iso-octyl acrylate, iso-octyl methacrylate, acrylic acid, methacrylic acid, itaconic acid, crotonic acid, O-methyl styrene, vinyl naphthalene, vinyl toluene, chloromethyl styrene, hydroxyethyl (meth)acrylate, hydroxypropyl (meth)acrylate, acrylonitrile, glycidyl methacrylate, acetoacetoxyethyl methacrylate, acetoacetoxy ethyl acrylate, vinyl chloride, vinylidene chloride, vinyl acetate, butyl acrylamide, ethyl acrylamide, 2-hydroxyethyl methacrylate phosphate and the like.

Latex emulsion polymers are well known in the art of coating compositions, and we do not intend the term to be especially limiting, although some latex emulsion polymers may be better suited as coating compositions, either inherently or in combination with the coalescents of the invention. Examples of commercial latex emulsion polymers useful according to the invention include Rhoplex SG-30, Rhoplex HG-74P, Rhoplex SG-10M, Rhoplex AC2508, Ucar 626, and Ucar 379G (all available from The Dow Chemical Company), Acronal 296D (BASF Corp.), Aquamac 705 and Aquamac 588 (Hexion Specialty Chemicals), and the like.

In one embodiment, the polymer is a latex polymer, and the latex polymers useful according to the invention may be a homopolymer, or a copolymer of an ethylenically unsaturated monomer and one or more additional copolymerizable monomers.

The latex emulsion polymers useful according to the invention are addition polymers that may be formed via a free radical addition polymerization. In such addition polymers, the propagating species may be a free radical, and the polymer is formed in a chain-growth fashion polymerization as understood in the art. As noted, these polymers are latex emulsion polymers in which a monomer solution may be emulsified in an aqueous solution, and under agitation reacted via a free-radical polymerization process as described herein, to form latex particles.

The water-based latexes useful according to the invention may generally be prepared by polymerizing acrylic (ethylenically unsaturated) monomers. Before conducting polymerization, these ethylenically unsaturated monomers are either pre-emulsified in water/surfactant mixture or used as such.

The polymerization process of making these ‘acrylic’ latexes may also require an initiator (oxidant), a reducing agent, or a catalyst. Suitable initiators include conventional initiators such as ammonium persulfate, sodium persulfate, hydrogen peroxide, t-butyl hydroperoxide, ammonium or alkali sulfate, di-benzoyl peroxide, lauryl peroxide, di-tertiarybutylperoxide, 2,2-azobisisobutyronitrile, benzoyl peroxide, and the like.

Suitable reducing agents are those which increase the rate of polymerization and include, for example, sodium bisulfite, sodium hydrosulfite, sodium formaldehyde sulfoxylate, ascorbic acid, isoascorbic acid, and mixtures thereof.

Suitable catalysts are those compounds which promote decomposition of the polymerization initiator under the polymerization reaction conditions thereby increasing the rate of polymerization. Suitable catalysts include transition metal compounds and driers. Examples of such catalysts include, but are not limited to, AQUACATO, ferrous sulfate heptahydrate, ferrous chloride, cupric sulfate, cupric chloride, cobalt acetate, cobaltous sulfate, and mixtures thereof.

The latex polymers of the invention are prepared from monomers characterized as being ethylenically unsaturated monomers that can participate in addition polymerization reactions. As used herein, ethylenically unsaturated monomers may also be described as vinyl monomers. The polymers made from such monomers are addition polymers, and may be formed as emulsion polymers, also known as latexes or latex emulsions.

The latex polymers useful according to the invention may have pendant moieties, meaning that the ethylenically unsaturated monomers used to prepare the latex polymers of the invention have been reacted into an addition polymer, and that a portion of the monomers remains as a pendant moiety. Alternatively, we may say that the polymers according to the invention have residues from the ethylenically unsaturated monomers of the invention, in which case we mean that the monomers have been reacted into an addition polymer via their ethylenic unsaturation, and that a portion of the monomers remains as a residue. Both these descriptions are well-known in the art of addition polymers, and the descriptions are not otherwise intended to be especially limiting.

The invention relates to the use of emulsion polymers which are also known as latexes, or as used herein, latex emulsions. In these latexes, the polymers formed may have a particle size ranging, for example, from about 80 nm to about 300 nm, or from 100 nm to 250 nm, or from 125 nm to 200 nm. The Te of such latexes may range, for example, from about 0° C. to about 80° C., or from 15° C. to 60° C., or from 20° C. to 40° C.

The latex polymers useful according to the invention may be prepared by an emulsion free-radical polymerization of ethylenically unsaturated monomers. These latex polymers may be homopolymers, or may be copolymers formed from more than one ethylenically unsaturated monomer.

Examples of ethylenically unsaturated monomers include, but are not limited to, acrylic and methacrylic acid esters such as methyl (meth)acrylate, ethyl (meth)acrylate, propyl (meth)acrylate, isopropyl (meth)acrylate, butyl (meth)acrylate, isobutyl (meth)acrylate, hexyl (meth)acrylate, isooctyl (meth)acrylate, isodecyl (meth)acrylate, lauryl (meth)acrylate, stearyl (meth)acrylate, phenoxyethyl (meth)acrylate, methoxyethyl (meth)acrylate, benzyl (meth)acrylate, ethoxyethyl (meth)acrylate, 2-ethylhexyl (meth)acrylate, cyclopentyl (meth)acrylate and isobutyl (meth)acrylate, as well as combinations of these monomers. A combination of these monomers may be used in order to achieve an appropriate Tg or other properties for the latex emulsion polymer.

Such acrylic and methacrylic acid esters having a C1-C20alcohol moiety are commercially available or can be prepared by known esterification processes. The acrylic and methacrylic acid ester may contain additional functional groups, such as, hydroxyl, amine, halogen, ether, carboxylic acid, amide, nitrile, and alkyl group. Such esters include carbodiimide (meth)acrylate, methyl (meth)acrylate, ethyl (meth)acrylate, butyl (meth)acrylate, isobutyl (meth)acrylate, ethylhexyl (meth)acrylate, octyl (meth)acrylate, isobutyl (meth)acrylate, allyl (meth)acrylate, and glycidyl (meth)acrylate.

Additional suitable polymerizable ethylenically unsaturated monomers include styrenic monomers. Styrenic monomers include styrene, as well as substituted styrenes such as C1-C6 alkyl ring-substituted styrene, C1-C3 alkyl alpha-substituted styrene or a combination of ring and an alpha-alkyl substituted styrene. Such styrenic polymerizable monomers include styrene, p-methyl styrene, o-methyl styrene, p-butyl styrene, alpha-methyl styrene, and combinations thereof.

In addition, vinyl esters may be used as copolymerizable mono-ethylenically unsaturated monomers, including vinyl esters of vinyl alcohol such as the VEOVA series available from Shell Chemical Company as VEOVA 5, VEOVA 9, VEOVA 10, and VEOVA 11 products. See O. W. Smith, M. J. Collins, P. S. Martin, and D. R. Bassett, Prog. Org. Coatings 22, 19 (1993).

In general, the vinyl monomers may be polymerized by a conventional emulsion free-radical initiated polymerization technique. The polymerization can be initiated by a water soluble or water-dispersible free-radical initiator, optionally in combination with a reducing agent, at an appropriate temperature, for example from 55 to 90° C. The polymerization of the monomers may be conducted batch wise, semi-batch, or in a continuous mode.

A conventional surfactant or a combination of surfactants may be used such as anionic or non-ionic emulsifier in the suspension or emulsion polymerization to prepare a polymer of the invention. Examples of such surfactants include, but are not limited to, alkali or ammonium alkylsulfate, alkylsulfonic acid, or fatty acid, oxyethylated alkylphenol, or any combination of anionic or non-ionic surfactant. A surfactant monomer may be used such as HITENOL HS-20 (which is a polyoxyethylene alkylphenyl ether ammonium sulfate available from DKS International, Inc., Japan). A list of surfactants is available in the treatise: McCutcheon's Emulsifiers & Detergents, North American Edition and International Edition, MC Publishing Co., Glen Rock, N.J. 1993. The amount of the surfactant used is usually between 0.1 to 6 wt %, based on the total weight of the monomers.

As polymerization initiators, any conventional free-radical initiator may be used such as hydrogen peroxide, t-butylhydroperoxide, ammonium or alkali sulfate, di-benzoyl peroxide, lauryl peroxide, di-tertiarybutylperoxide, 2,2′-azobisisobutyronitrile, benzoyl peroxide, and the like. The amount of the initiator is typically between 0.05 to 6.0 wt %, based on the total weight of the total monomers. A free-radical initiator may be combined with a reducing agent to form a redox initiating system. Suitable reducing agents are those which increase the rate of polymerization and include, for example, sodium bisulfite, sodium hydrosulfide, sodium, ascorbic acid, isoascorbic acid and mixtures thereof. The redox initiating system can be used at similar levels as the free-radical initiators.

In addition, in combination with the initiators and reducing agents, polymerization catalysts may be used. Polymerization catalysts are those compounds which increase the rate of polymerization by promoting decomposition of the free radical initiator in combination with the reducing agent at the reaction conditions. Suitable catalysts include transition metal compounds such as, for example, ferrous sulfate heptahydrate, ferrous chloride, cupric sulfate, cupric chloride, cobalt acetate, cobaltous sulfate, and mixtures thereof.

In addition, a low level of a chain transfer agent may also be used to prepare a latex polymer useful in accordance with the invention. Suitable chain transfer agents include, but are not limited to, butyl mercaptan, n-octylmercaptan, n-dodecyl mercaptan, butyl or methyl mercaptopropionate, mercaptopropionic acid, 2-ethylhexyl 3-mercaptopropionate, n-butyl 3-mercaptopropionate, isodecylmercaptan, octadecylmercaptan, mercaptoacetic acid, haloalkyl compounds, (such as carbon tetrabromide and bromodichoromethane), and the reactive chain transfer agents described in U.S. Pat. No. 5,247,040, incorporated herein by reference. In particular, mercaptopropionate, allyl mercaptopropionate, allyl mercaptoacetate, crotyl mercaptopropionate and crotyl mercaptoacetate, and mixtures thereof, represent preferred chain transfer agents.

A copolymerizable monomer known to promote wet adhesion may also be incorporated into the polymer. Examples of wet adhesion promoting monomers include, but are not limited to, nitrogen-containing monomers such as t-butylaminoethyl methacrylate, dimethylaminoethyl methacrylate, diethylaminoethyl methacrylate, N,N-dimethylaminopropylmethacrylamide, 2-t-butylaminoethyl methacrylate, N,N dimethylaminoethyl acrylate and N-(2-methacryloyloxy ethyl)ethylene urea.

Water-dispersible and water-soluble polymers may also be employed as surfactants or stabilizers in accordance with the present invention. Examples of such polymeric stabilizers include water-dispersible polyesters as described in U.S. Pat. Nos. 4,946,932 and 4,939,233; water-dispersible polyurethanes as described in U.S. Pat. Nos. 4,927,876 and 5,137,961; and alkali-soluble acrylic resins as described in U.S. Pat. No. 4,839,413. Cellulosics and polyvinyl alcohols may also be used.

Surfactants and stabilizers may be used during the polymerization to control, for example, particle nucleation and growth, particle size and stability or they may be post-added to enhance stability of the latex or to modify other properties of the latex such as surface tension, wettability, and the like.

At least one ethylenically unsaturated copolymerizable surfactant may be employed, for example those possessing isopropenyl phenyl or allyl groups. Copolymerizable surfactants may be anionic, such as containing a sulfate or sulfonate group, or nonionic surfactants. Other copolymerizable surfactants include those containing polyoxyethylene alkyl phenyl ether moieties. Additional copolymerizable surfactants include sodium alkyl allyl sulfosuccinate.

The latex polymers in accordance with the invention may have a weight average molecular weight (Mw), for example, of from 1,000 to 1,000,000, as determined by gel permeation chromatography (GPC), or from 5,000 to 250,000.

The particle size for the aqueous dispersions in accordance with the invention may be, for example, from about 0.01 to about 25 μm, or from 0.05 to 1 μm, or from 0.075 to 500 μm. In an emulsion polymerization in accordance with the invention, the particle size of the latex may range, for example, from 0.01 to 5 μm.

The latex particles generally have a spherical shape, and the spherical polymeric particles may have a core portion and a shell portion or a gradient structure. The core/shell polymer particles may also be prepared in a multi-lobe form, a peanut shell, an acorn form, a raspberry form, or any other form. If the particles have a core/shell structure, the core portion may comprise from about 20 to about 80 wt % of the total weight of the particle, and the shell portion may comprise about 80 to about 20 wt % of the total weight of the particle.

The glass transition temperature (Tg) of the latex polymers in accordance with the present invention, in the absence of the coalescents described herein, may be up to about 100° C. In a preferred embodiment of the present invention, where a film forming at ambient temperatures of the particles is desirable, the glass transition temperature of the polymer itself may preferably be under 60° C.

The latex polymers of the invention may comprise enamine functional polymers, with the enamine functionality serving to improve the hydrolytic stability of the acetoacetoxy group. Enamine functional polymers have been described in Polymer Bulletin 32, 419-426 (1994). Additionally, enamine functional polymers are described in European Patent Application No. 0492847 A2; U.S. Pat. Nos. 5,296,530; and 5,484,849, all of which are incorporated herein by reference.

The coating compositions of the invention may further comprise other components commonly used in paint formulations, such as, for example, pigments, filler, rheology modifiers, thickeners, wetting and dispersing agents, deformers, freeze-thaw additives, colorants, open-time additives, driers, catalysts, crosslinkers, biocides, light stabilizers, and the like.

The driers are capable of promoting oxidative crosslinking of the unsaturated moieties and providing enhanced coating properties. Examples of commercial driers include Zirconium Hex-Cem®, Cobalt Ten-Cem®, calcium Cem-AII®, Zirconium Hydro-Cem, and Cobalt Hydro-Cure® II sold by OMG Americas of West-Lake, Ohio. Examples of driers based on unsaturated fatty alcohols include oleyl alcohol, linoleoyl alcohol, geraniol, or citronellol.

In one embodiment, the composition has a minimum film formation temperature in the range of from about −35° C. to about 60° C. In one embodiment, the composition has a minimum film formation temperature in the range of from about −35° C. to about 5° C. In one embodiment, the composition has a minimum film formation temperature in the range of from about −35° C. to about 60° C. In one embodiment, the composition has a minimum film formation temperature in the range of from about −35° C. to about 2° C.

The minimum film formation temperature of a latex is the lowest temperature at which the latex forms a practical film. MFFT can be measured using ASTM D2354. The efficiency of a coalescent can be determined by determining the amount of the coalescent required to reduce the MFFT of a latex polymer to 4.4° C., which is the lowest desirable application temperature of a paint. It is generally considered unacceptable if the amount of the coalescent present in a paint formulation exceeds 20% by weight based on the solids of the latex polymer. This is particularly important for a non-volatile coalescent since the coalescent will remain in the dried film and cause a detrimental effect on the coating properties such as, for example, hardness, scrub resistance, and block resistance. As shown in the Table 1, the level of coalescent in phr required to lower the MFFT of a variety of latex resins is less than 7 phr at 4.4° C. and less than 8.5 phr at 1.67° C., exemplifying the coalescent efficiency of these materials.

In one embodiment, when R¹ is hydrogen, R² is not ethyl, hexyl or decyl, and wherein when R¹ is methyl, R² is not methyl.

In one embodiment, the compound of Formula I is chosen from

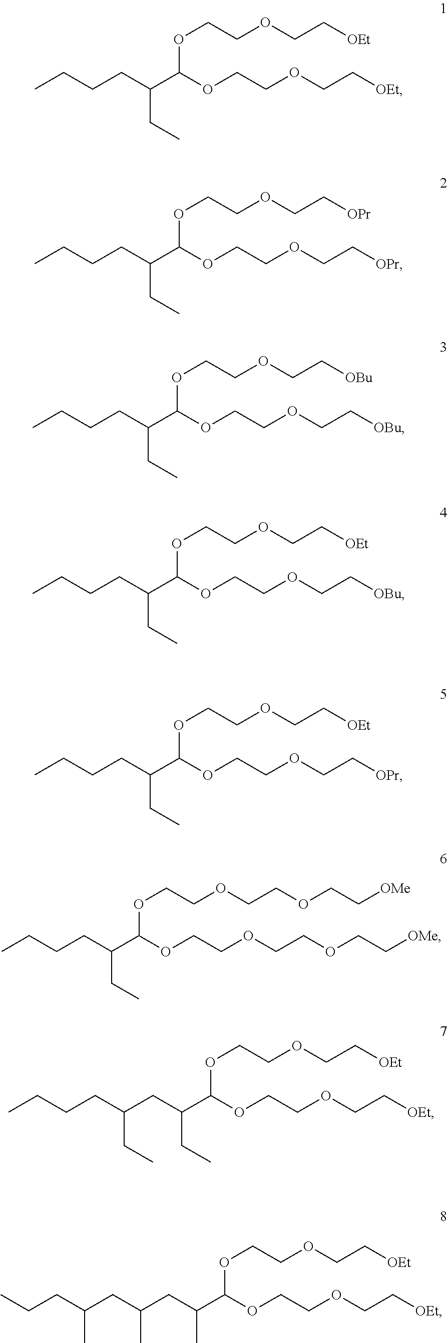

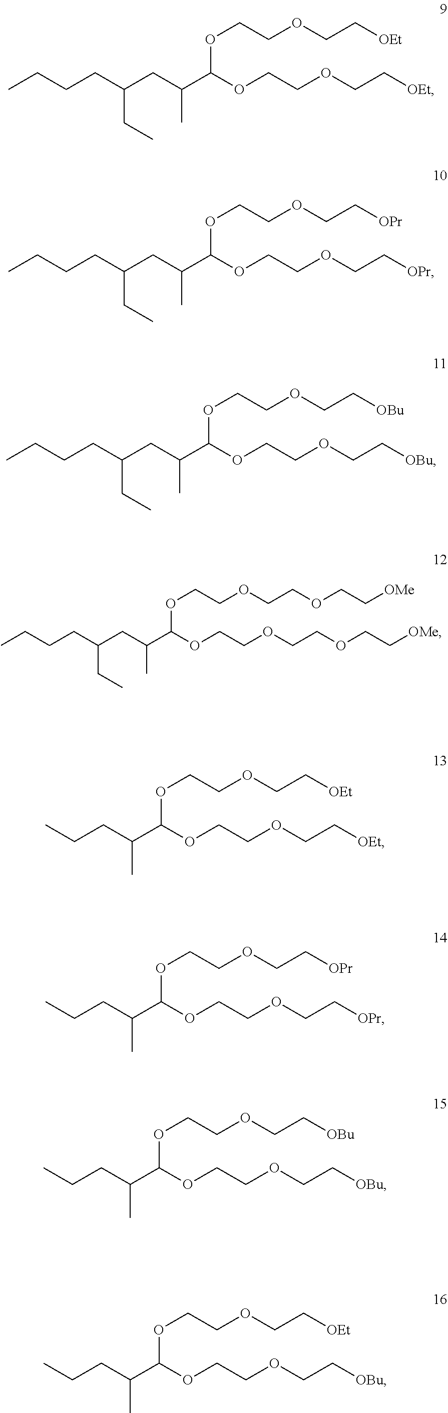

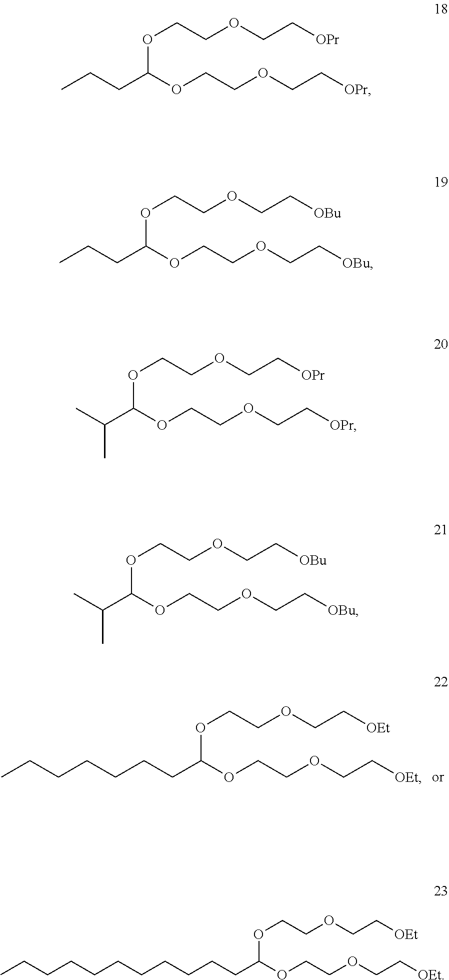

In one class of this embodiment, the compound of Formula I is

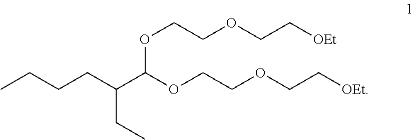 [SMILES: CCCCC(CC)C(OCCOCCOCC)OCCOCCOCC]

In one class of this embodiment, the compound of Formula I is

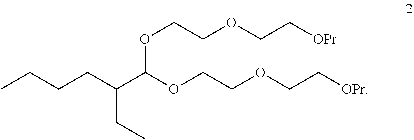

In one class of this embodiment, the compound of Formula I is

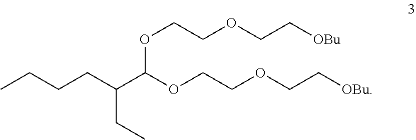

In one class of this embodiment, the compound of Formula I is

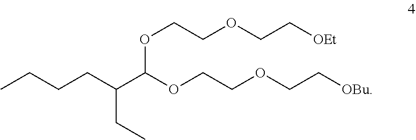

In one class of this embodiment, the compound of Formula I is

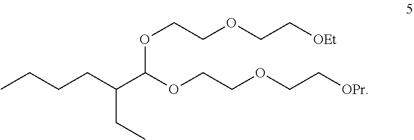

In one class of this embodiment, the compound of Formula I is

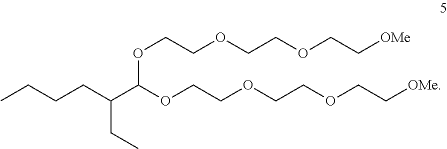 [SMILES: CCCCC(CC)C(OCCOCCOCCOC)OCCOCCOCCOC]

In one class of this embodiment, the compound of Formula I is

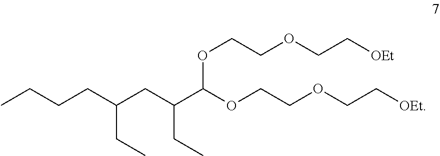 [SMILES: CCCCC(CC)CC(CC)C(OCCOCCOCC)OCCOCCOCC]

In one class of this embodiment, the compound of Formula I is

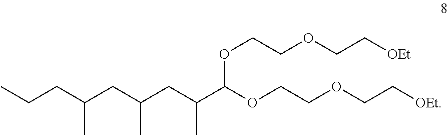 [SMILES: CCCC(C)CC(C)CC(C)C(OCCOCCOCC)OCCOCCOCC]

In one class of this embodiment, the compound of Formula I is

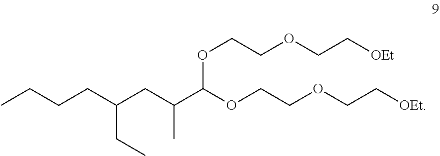 [SMILES: CCCCC(CC)CC(C)C(OCCOCCOCC)OCCOCCOCC]

In one class of this embodiment, the compound of Formula I is

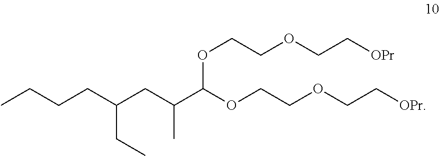

In one class of this embodiment, the compound of Formula I is

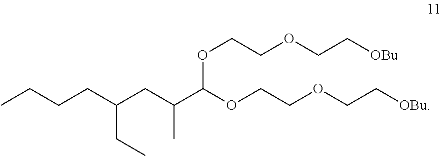

In one class of this embodiment, the compound of Formula I is

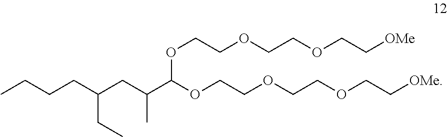 [SMILES: CCCCC(CC)CC(C)C(OCCOCCOCCOC)OCCOCCOCCOC]

In one class of this embodiment, the compound of Formula I is

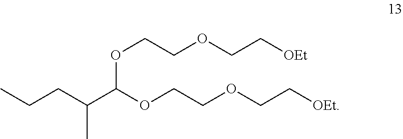 [SMILES: CCCC(C)C(OCCOCCOCC)OCCOCCOCC]

In one class of this embodiment, the compound of Formula I is

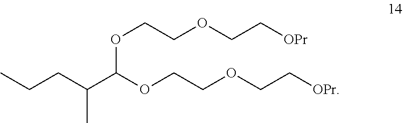

In one class of this embodiment, the compound of Formula I is

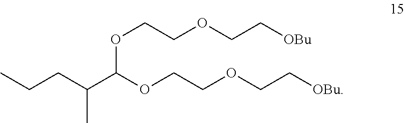

In one class of this embodiment, the compound of Formula I is

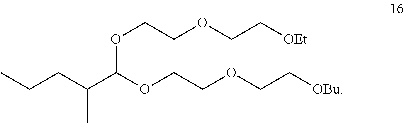

In one class of this embodiment, the compound of Formula I is

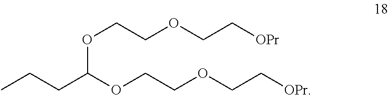

In one class of this embodiment, the compound of Formula I is

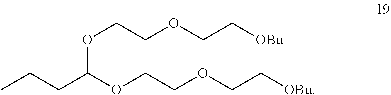

In one class of this embodiment, the compound of Formula I is

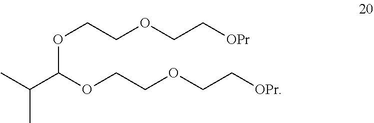

In one class of this embodiment, the compound of Formula I is

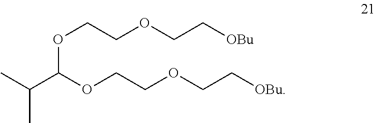

In one class of this embodiment, the compound of Formula I is

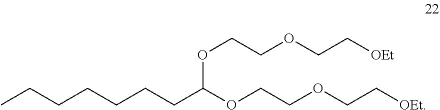 [SMILES: CCCCCCCC(OCCOCCOCC)OCCOCCOCC]

In one class of this embodiment, the compound of Formula I is

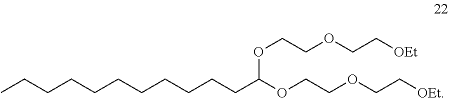 [SMILES: CCCCCCCCCCCC(OCCOCCOCC)OCCOCCOCC]

### EXAMPLES

This invention can be further illustrated by the following examples thereof, although it will be understood that these examples are included merely for purposes of illustration and are not intended to limit the scope of the invention unless otherwise specifically indicated.

### Abbreviations

mL is milliliter; wt % is weight percent; eq is equivalent(s); hrs or h is hour(s); mm is millimeter; m is meter; GC is gas chromatography; ° C. is degree Celsius; min is minute; t_{R} is retention time; Et is ethyl; J is coupling constant; H is hydrogen; 1H is proton; NMR is nuclear magnetic resonance; MHz is megahertz; DMSO-d6 is hexadeuterated dimethyl sulfoxide; t is triplet; mult is multiplet; d is doublet; Hz is hertz; MPEG is methyl polyethylene glycol; p-TSA is p-toluene sulfonic acid; g is gram; mmol is millimole; mol is mole; kg is kilogram; L is liter; Bu is butyl; Pr is propyl; MeP is methyl palmitate; w/v is weight/volume; μL is microliter; Tg is glass transition temperature; MFFT is minimum film-forming temperature; phr is parts per hundred resin; MW is molecular weight.

### Synthesis of Prototype Acetals:

The C11 and C12 branched aldehyde intermediate were synthesized from propionaldehyde or butyraldehyde by crossed aldol chemistry using standard methods.

### General Procedure (Exceptions Otherwise Noted):

An aldehyde was mixed with a glycol ether solvent (10 eq). Amberlyst™ 15 (10 wt % based on aldehyde) was thoroughly washed with the glycol ether solvent prior to use and then added to the mixture of aldehyde and glycol ether. The reaction mixture was stirred for 24-48 h. Typically, conversion reached ˜50% to give the desired acetal. Ethyl acetate, heptane, or diethyl ether (250 mL) were added to dilute the reaction mixture, which was then washed with saturated aq. NaHCO₃ soln (250 mL). Additional water was added to achieve complete phase separation. The organic phase was dried with MgSO₄ and then filtered through a 1 micron glass fiber disc. The solution was concentrated in vacuo. The crude product was purified by Kugelrohr distillation at 2 mm Hg and 100-120° C. to remove unreacted aldehyde and excess glycol ether to obtain the desired acetals in high yield.

GC analysis was conducted using the following method: 30 m×0.25 mm DB-5 column, 100° C. for 3 min, 100 to 300° C. at 25° C./min, 300° C. for 14 min. Methyl palmitate t_{R}=10.15 min.

### Example 1: 10-(heptan-3-yl)-3,6,9,11,14,17-hexaoxanonadecane

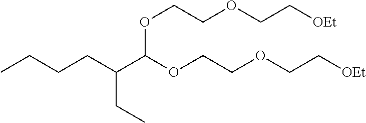 [SMILES: CCCCC(CC)C(OCCOCCOCC)OCCOCCOCC]

Yield: 54%. GC (t_{R})=11.69 min, ¹H NMR (500 MHz, DMSO-d6) δ=4.37 (d, J=6 Hz, 1H), 3.67-3.62 (mult., 2H), 3.57-3.50 (m, 10H), 3.49-3.46 (m, 4H), 3.43 (q, 4H), 1.51-1.36 (m, 3H), 1.31-1.18 (m, 6H), 1.10 (t, 6H), 0.89-0.82 (m, 6H).

### Example 2: 11-(heptan-3-yl)-4,7,10,12,15,18-hexaoxahenicosane

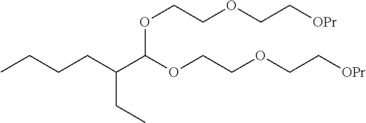

Yield: 38%. GC (t_{R})=12.12 min, ¹H NMR (500 MHz, DMSO-d6) δ=4.37 (d, J=5.5 Hz, 1H), 3.68-3.62 (m, 2H), 3.57-3.51 (m, 10H), 3.49-3.46 (m, 4H), 3.34 (t, 4H), 1.50 (sextet, 4H), 1.45-1.36 (m, 3H), 1.30-1.19 (m, 6H), 0.89-0.82 (m, 12H).

### Example 3: 12-(heptan-3-yl)-5,8,11,13,16,19-hexaoxatricosane

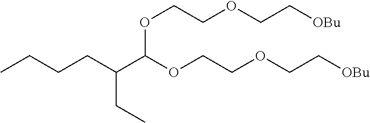

Yield: 35%. GC (t_{R})=13.33 min, ¹H NMR (500 MHz, DMSO-d6) δ=4.37 (d, J=6 Hz, 1H), 3.68-3.61 (m, 2H), 3.56-3.50 (m, 10H), 3.49-3.45 (m, 4H), 3.38 (t, 4H), 1.50-1.37 (m, 6H), 1.36-1.17 (m, 10H), 0.90-0.82 (m, 13H)

### Example 4: 1:2:1 Mixture of 10-(heptan-3-yl)-3,6,9,11,14,17-hexaoxanonadecane, 10-(heptan-3-yl)-3,6,9,11,14,17-hexaoxahenicosane, and 12-(heptan-3-yl)-5,8,11,13,16,19-hexaoxatricosane

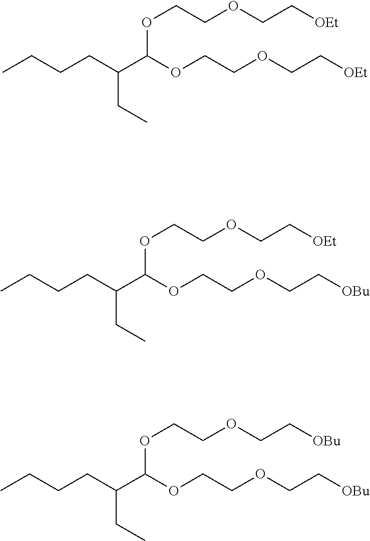

Yield: 42%. GC (t_{R})=11.37, 12.12, 12.88 min (1:2:1 ratio), ¹H NMR (500 MHz, DMSO-d6) δ=4.37 (d, J=6 Hz, 1H), 3.68-3.62 (m, 2H), 3.57-3.50 (m, 10H), 3.49-3.45 (m, 4H), 3.43 (q, 2H), 3.38 (t, 2H), 1.50-1.37 (m, 5H), 1.36-1.19 (m, 7H), 1.10 (t, 3H), 0.91-0.82 (m, 10H).

### Example 5: 1:2:1 Mixture of 10-(heptan-3-yl)-3,6,9,11,14,17-hexaoxanonadecane, 10-(heptan-3-yl)-3,6,9,11,14,17-hexaoxaicosane, and 11-(heptan-3-yl)-4,7,10,12,15,18-hexaoxahenicosane

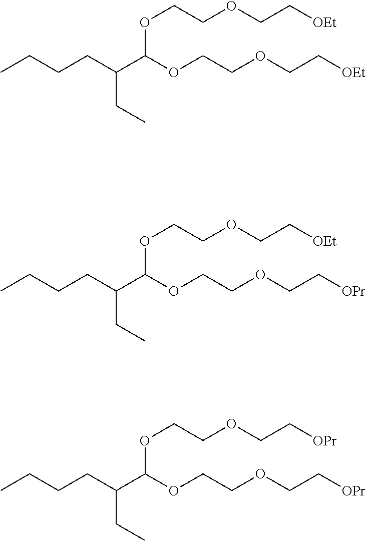

Yield: 45%. GC (t_{R})=11.37, 11.75, 12.11 min (1:2:1 ratio), ¹H NMR (500 MHz, DMSO-d6) δ=4.37 (d, J=7 Hz, 1H), 3.65 (m, 2H), 3.56-3.50 (m, 10H), 3.49-3.46 (m, 4H), 3.43 (q, 2H), 3.34 (t, 2H), 1.54-1.37 (m, 6H), 1.31-1.17 (m, 6H), 1.10 (t, 3H), 0.89-0.82 (m, 10H).

### Example 6: 12-(heptan-3-yl)-2,5,8,11,13,16,19,22-octaoxatricosane

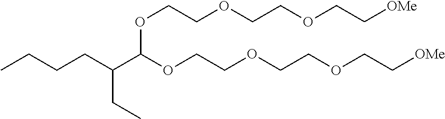 [SMILES: CCCCC(CC)C(OCCOCCOCCOC)OCCOCCOCCOC]

To a 2 L round-bottom flask was added 100 g (780 mmol) of 2-ethylhexanal and 1.281 kg (7.799 mol) of MPEG 165. 10 g of Amberlyst™ 15 was added. After 1.5 hrs, 5 g of p-TSA was added. The reaction was stirred for an additional 28.5 h. The mixture was diluted with ethyl acetate (250 mL) and washed with 500 mL of saturated sodium bicarbonate solution. The layers were separated. The aqueous component was extracted with toluene (250 mL). The organics were combined and dried with MgSO₄. After filtration, the volatiles were removed under reduced pressure using a rotary evaporator. The crude oil was then Kugelrohr distilled at 2 mm Hg and 100-120° C. to remove unreacted aldehyde and excess MPEG 165 to obtain the desired product as a colorless oil. Yield: 38%. GC (t_{R})=13.55 min, ¹H NMR (500 MHz, DMSO-d6) δ=4.37 (d, J=5.5 Hz, 1H), 3.65 (m, 2H), 3.57-3.49 (m, 17H), 3.45-3.41 (m, 5H), 3.25 (s, 6H), 1.52-1.36 (3H), 1.31-1.17 (m, 6H), 0.90-0.82 (m, 6H).

### Example 7: 10-(5-ethylnonan-3-yl)-3,6,9,11,14,17-hexaoxanonadecane

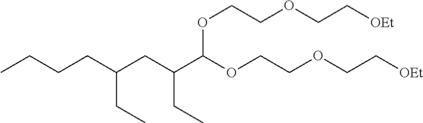 [SMILES: CCCCC(CC)CC(CC)C(OCCOCCOCC)OCCOCCOCC]

To a 1 L round-bottom flask was added 50 g (373 mmol) of 2,4-diethyloctenal and 927 g (5.03 mol) of diethylene glycol mono-ethyl ether (“DB solvent”). 7.09 g (37.3 mmol) of p-TSA was added. The reaction was stirred for 20 hrs. At that time, GC analysis indicated 51.2% conversion to the acetal. The mixture was then poured into 1 L of saturated sodium bicarbonate and then extracted with heptane (200 mL). The layers were separated, and the aqueous component was back-extracted with heptane (300 mL). The organics were combined and then dried with MgSO₄. After filtration, the volatiles were stripped under reduced pressure using a rotary evaporator. The crude oil was then Kugelrohr distilled at 2 mm Hg and 100-120° C. to remove unreacted aldehyde and excess DB solvent to obtain the desired product as a colorless oil. GC (t_{R})=12.88 min, ¹H NMR (500 MHz, DMSO-d6) δ=4.37 (d, J=5.5 Hz, 1H), 3.69-3.62 (m, 2H), 3.57-3.50 (m, 10H), 3.49-3.45 (m, 4H), 3.43 (q, 4H), 1.60-1.48 (m, 1H), 1.47-1.34 (m, 1H), 1.36-1.13 (m 12H), 1.10 (t, 6H), 0.90-0.79 (m 9H).

### Example 8: 10-(4,6-dimethylnonan-2-yl)-3,6,9,11,14,17-hexaoxanonadecane

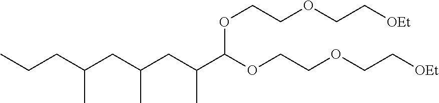 [SMILES: CCCC(C)CC(C)CC(C)C(OCCOCCOCC)OCCOCCOCC]

Yield: 23%. GC (t_{R})=12.44-12.68 min, ¹H NMR (500 MHz, DMSO-d6) δ=4.26-4.21 (m, 1H), 3.70-3.59 (m, 2H), 3.57-3.49 (m, 10H), 3.50-3.45 (m, 4H), 3.43 (q, 4H), 1.78-1.69 (m, 1H), 1.64-1.17 (m, 5H), 1.10 (t, 6H), 1.08-0.89 (m, 5H), 0.88-0.75 (m, 12H).

### Example 9: 10-(4-ethyloctan-2-yl)-3,6,9,11,14,17-hexaoxanonadecane

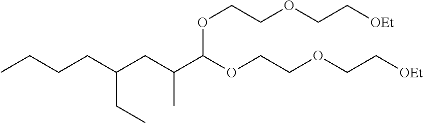 [SMILES: CCCCC(CC)CC(C)C(OCCOCCOCC)OCCOCCOCC]

Yield: 43%. GC (t_{R})=12.32 min, ¹H NMR (500 MHz, DMSO-d6) δ=4.23 (m, 1H), 3.68-3.60 (m, 2H), 3.57-3.50 (m, 10H), 3.49-3.45 (m, 4H), 3.43 (q, 4H), 1.70 (m, 1H), 1.41-1.13 (m, 8H), 1.10 (t, 6H), 1.02-0.91 (m, 1H), 0.89-0.85 (m, 3H), 0.84-0.78 (m, 9H).

### Example 10: 11-(4-ethyloctan-2-yl)-4,7,10,12,15,18-hexaoxahenicosane

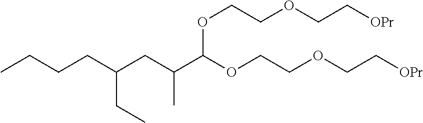

Yield: 50%. GC (t_{R})=13.13 min, ¹H NMR (500 MHz, DMSO-d6) δ=4.23 (m, 1H), 3.69-3.60 (m, 2H), 3.56-3.50 (m, 10H), 3.49-3.45 (m, 4H), 3.34 (t, 4H), 1.71 (m, 1H), 1.50 (sextet, 4H), 1.40-1.09 (m, 12H), 0.96 (m, 1H), 0.90-0.78 (m, 15H).

### Example 11: 12-(4-ethyloctan-2-yl)-5,8,11,13,16,19-hexaoxatricosane

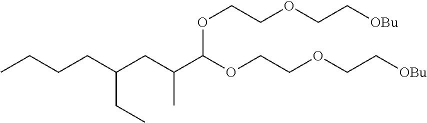

Yield: 27%. GC (t_{R})=14.07 min, ¹H NMR (500 MHz, DMSO-d6) δ=4.23 (m, 1H), 3.64 (m, 2H), 3.57-3.49 (m, 10H), 3.48-3.45 (m, 4H), 3.38 (t, 4H), 1.71 (m, 1H), 1.47 (m, 4H), 1.40-1.08 (m, 14H), 0.96 (m, 1H), 0.88 (m, 9H), 0.84-0.78 (m, 6H).

### Example 12: 12-(4-ethyloctan-2-yl)-2,5,8,11,13,16,19,22-octaoxatricosane

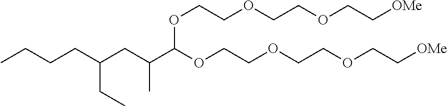 [SMILES: CCCCC(CC)CC(C)C(OCCOCCOCCOC)OCCOCCOCCOC]

Yield: 46%. GC (t_{R})=14.53 min, ¹H NMR (500 MHz, DMSO-d6) δ=4.23 (m, 1H), 3.63 (m, 2H), 3.55-3.49 (m, 18H), 3.44 (m, 4H), 3.25 (s, 6H), 1.71 (m, 1H), 1.39-1.06 (m, 11H), 0.95 (m, 1H), 0.88 (m, 3H), 0.84-0.78 (m, 6H).

### Example 13: 10-(pentan-2-yl)-3,6,9,11,14,17-hexaoxanonadecane

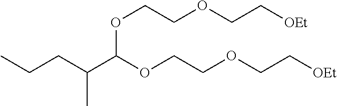 [SMILES: CCCC(C)C(OCCOCCOCC)OCCOCCOCC]

Yield: 58%. GC (t_{R})=10.77 min, ¹H NMR (500 MHz, DMSO-d6) δ=4.24 (d, J=6 Hz, 1H), 3.68-3.61 (m, 2H), 3.56-3.50 (m, 10H), 3.49-3.45 (m, 5H), 3.44 (q, 4H), 1.67 (m, 1H), 1.49-1.17 (m, 4H), 1.10 (t, 3H), 1.05 (m, 2H), 0.86 (t, 3H), 0.83 (d, 3H).

### Example 14: 11-(pentan-2-yl)-4,7,10,12,15,18-hexaoxahenicosane

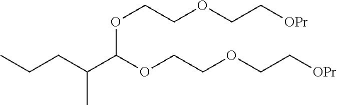

Yield: 36%. GC (t_{R})=11.54 min, ¹H NMR (500 MHz, DMSO-d6) δ=4.24 (d, J=6 Hz, 1H), 3.65 (m, 2H), 3.56-3.51 (m, 10H), 3.49-3.46 (m, 4H), 3.34 (t, 4H), 1.66 (m, 1H), 1.50 (sextet, 4H), 1.45-1.30 (m, 2H), 1.22 (m, 1H), 1.03 (m, 1H), 0.86 (t, 9H), 0.83 (d, 3H).

### Example 15: 12-(pentan-2-yl)-5,8,11,13,16,19-hexaoxatricosane

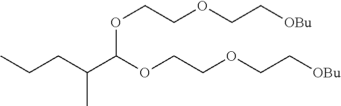

Yield: 52%. GC (t_{R})=12.34 min, ¹H NMR (500 MHz, DMSO-d6) δ=4.24 (d, J=6.0 Hz, 1H), 3.64 (m, 2H), 3.54 (m, 10H), 3.46 (m, 4H), 3.38 (t, 6H), 1.65 (m, 1H), 1.47 (m, 4H), 1.31 (m, 5H), 1.22 (m, 1H), 1.02 (m, 1H), 0.88 (t, 6H), 0.86 (t, 3H), 0.83 (d, 3H).

### Example 16: 1:2:1 Mixture of 10-(pentan-2-yl)-3,6,9,11,14,17-hexaoxanonadecane, 10-(pentan-2-yl)-3,6,9,11,14,17-hexaoxahenicosane, and 12-(pentan-2-yl)-5,8,11,13,16,19-hexaoxatricosane

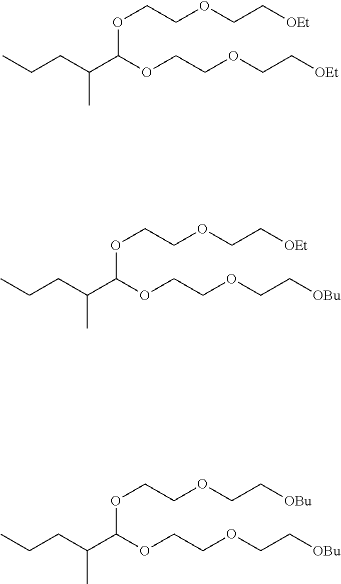

Yield: 37%. GC (t_{R})=10.76, 11.50, and 12.25 min (1:2:1 ratio), ¹H NMR (500 MHz, DMSO-d6) δ=4.24 (d, J=6 Hz, 1H), 3.65 (m, 2H), 3.56-3.49 (m, 10H), 3.50-3.45 (m, 4H), 3.43 (q, 2H), 3.39 (t, 2H), 1.67 (m, 1H), 1.50-1.19 (m, 8H), 1.10 (t, 2H), 1.04 (m, 1H), 0.88-0.82 (m, 9H).

### Example 17: 10-propyl-3,6,9,11,14,17-hexaoxanonadecane

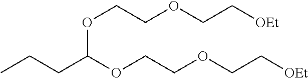 [SMILES: CCCC(OCCOCCOCC)OCCOCCOCC]

Yield: 48%. GC (t_{R})=10.28 min, ¹H NMR (500 MHz, DMSO-d6) δ=4.53 (t, J=5.5 Hz, 1H), 3.62 (m, 2H), 3.55-3.49 (m, 9H), 3.49-3.46 (m, 4H), 3.43 (q, 4H), 1.50 (m, 2H), 1.32 (m, 2H), 0.95 (m, 1H), 1.10 (t, 6H), 0.88 (t, 3H).

### Example 18: 11-propyl-4,7,10,12,15,18-hexaoxahenicosane

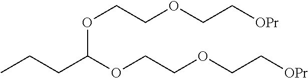

Yield: 39%. GC (t_{R})=11.01 min, ¹H NMR (500 MHz, DMSO-d6) δ=4.53 (d, J=6 Hz, 1H), 3.62 (m, 2H), 3.54-3.49 (m, 10H), 3.47 (m, 4H), 3.34 (t, 4H), 1.50 (m, 6H), 1.31 (m, 2H), 0.89-0.84 (m, 9H).

### Example 19: 12-propyl-5,8,11,13,16,19-hexaoxatricosane

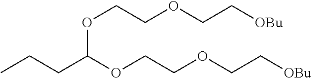

Yield: 60%. GC (t_{R})=11.79 min, ¹H NMR (500 MHz, DMSO-d6) δ=4.53 (d, J=5.5 Hz, 1H), 3.61 (m, 2H), 3.55-3.49 (m, 10H), 3.46 (m, 4H), 3.38 (t, 4H), 1.48 (m, 6H), 1.30 (m, 6H), 0.88 (t, 9H).

### Example 20: 11-isopropyl-4,7,10,12,15,18-hexaoxahenicosane

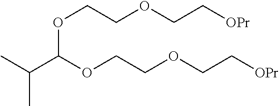

Yield: 42%. GC (t_{R})=10.88 min, ¹H NMR (500 MHz, DMSO-d6) δ=4.17 (d, J=6.5 Hz, 1H), 3.64 (m, 2H), 3.56-3.50 (m, 10H), 3.50-3.45 (m, 4H), 3.34 (t, 4H), 1.79 (m, 1H), 1.49 (sextet, 4H), 0.87-0.84 (m, 12H).

### Example 21: 12-isopropyl-5,8,11,13,16,19-hexaoxatricosane

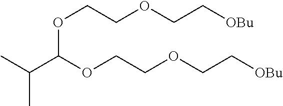

Yield: 49%. GC (t_{R})=11.69 min, ¹H NMR (500 MHz, DMSO-d6) δ=4.17 (d, J=6.5 Hz, 1H), 3.64 (m, 2H), 3.56-3.50 (m, 10H), 3.47 (m, 4H), 3.38 (t, 4H), 1.79 (sextet, 1H), 1.47 (m, 4H), 1.31 (m, 4H), 0.88 (t, 6H), 0.85 (d, 6H).

### Example 22: 10-octyl-3,6,9,11,14,17-hexaoxanonadecane

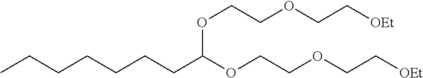 [SMILES: CCCCCCCC(OCCOCCOCC)OCCOCCOCC]

Yield: 63%. GC (t_{R})=11.96 min, ¹H NMR (500 MHz, DMSO-d6) δ=4.52 (t, J=5.5 Hz, 1H), 3.61 (m, 2H), 3.54-3.49 (m, 10H), 3.46 (m, 3H), 3.43 (q, 4H), 1.51 (m, 2H), 1.27 (bs, 10H), 1.10 (t, 6H), 0.87 (t, 3H).

### Example 23: 10-dodecyl-3,6,9,11,14,17-hexaoxanonadecane

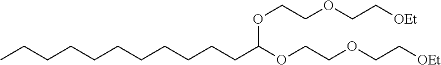 [SMILES: CCCCCCCCCCCC(OCCOCCOCC)OCCOCCOCC]

Yield: 48%. GC (t_{R})=13.86 min, ¹H NMR (500 MHz, DMSO-d6) δ=4.51 (t, J=6 Hz, 1H), 3.62 (m, 2H), 3.54-3.49 (m, 10H), 3.48-3.46 (m, 4H), 3.43 (q, 4H), 1.50 (m, 2H), 1.25 (bs, 20H), 1.10 (t, 6H), 0.86 (t, 3H).

### Volatility Screening (Volatile Organic Compound, VOC): ASTM D6886

Prototypes were required to pass a screening test for suitability in a low-VOC/low odor paint formulation. Volatility screening is done by GC and is based on ASTM D-6886, an internal standard method for determining weight percent VOCs in waterborne air-dry coatings. The GC conditions used are as follows:

GC: Agilent 6890 or equivalent; Column: DB-5 (5% phenyl/95% methylpolysiloxane); 30 m×0.25 mm ID×1.00 μm, Agilent Technologies, P/N: 22-5033; Injector: Split/splitless injector, 280° C., Split mode; Carrier Gas: Helium; Column Flow: Constant flow mode, 1.00 mL/minute; Linear Velocity: 25.45 cm/second (at initial oven temperature of 50° C.); Carrier Pressure: 11.96 psi (at initial oven temperature of 50° C.); Total Flow: 53.5 mL/minute; Split Ratio: 50:1

Septum Purge Flow: 2 mL/minute; Detector: Flame Ionization Detector (FID), 80° C.; Detector Gas Flows: Hydrogen: 40 mL/minute; Air: 400 mL/minute; Column+Makeup (Helium): 45 mL/minute; Oven Program: Initial Temperature: 50° C.; Initial Hold Time: 4 minutes; Program Rate-1: 20° C./minute; Final Temperature-1: 250° C.; Hold Time-1: 6 minutes; Program Rate-2: 20° C./minute; Final Temperature-2: 300° C.; Hold Time-2: 37.5 minutes; Total Run Time: 60 minutes; Data System: EZ-Chrom Elite, Version 3.3.2SP2 or equivalent; Injection Volume: 1.0 μL; Autoinjector: Shimadzu AOC-5000 or equivalent; Rinse Solvent: Acetonitrile.

The internal standard solution used for this method is 1.0265% (w/v) MeP in acetonitrile. It is prepared by accurately weighing 1.0265±0.005 g of MeP into a 100-mL volumetric flask and diluting to the mark with acetonitrile.

Prior to analyzing samples, a five-point calibration should be performed using Texanol™ standards that reflect the range of expected VOC concentrations (e.g., 1-10%). To prepare Texanol™ calibration standards, first tare a 4-dram vial and cap. Then, add the appropriate amount of Texanol™ for each standard based on a final weight of 0.7000 g (e.g., 0.0070 g for a 1% standard). Next, backfill the 4-dram vial with acetonitrile (or other appropriate solvent) until a final weight of 0.7000 is achieved. Add 9.0 mL of acetonitrile (or other suitable solvent), followed by 1000.0 μL of internal standard solution. Cap the vial and vortex thoroughly, then transfer a portion of the solution to a GC vial for injection. Repeat for all desired concentrations. The response factor generated by the Texanol™ calibration is used to quantify all VOCs eluting before methyl palmitate.

A reagent blank, containing all reagents except for the sample or standard, should be run before each set of samples to ensure the chromatographic system is free from interferences. Additionally, it is always prudent to prepare a control standard containing a known concentration of Texanol™ and run it before and after the samples. This is to confirm the validity of the calibration and ensure the instrument is functioning properly. Control standards are prepared like calibration standards, the procedure for which was described previously in this section. Ideally, the concentration of control standards should closely resemble the expected concentrations of VOCs contained in the samples.

Neat coalescent samples were prepared by the following procedure:
1. Accurately weigh 0.7000 g of sample into a tarred 4-dram vial with screw cap
2. Add 9.0-mL of acetonitrile (or other suitable solvent)
3. Accurately add 1000.0 μL of internal standard solution
4. Cap the vial and vortex thoroughly
5. Transfer a portion of the resulting solution to a GC vial for injection

### MFFT Screening: ASTM D2354-10e

MFFT efficiency testing is based on ASTM D2354. The model instrument that we use is an MFFT-90 bar which allows samples to be tested from −10° C. to 90° C. For waterborne latexes we are concerned about reaching a temperature of 2° C. To reach that temperature, we would set our MFFT bar to range from 0° C. to 18° C. The reason we test in this range is that T_{g} values for waterborne latexes somewhat correlate with their coinciding MFFT value. The higher the T_{g} value, the higher the MFFT value and vice versa. With that being said, neat commercial architectural latexes typically lie within this temperature range when testing for MFFT efficiency. Depending on the T_{g} of the material being tested, the range can be adjusted accordingly to determine the film's MFFT.

The ultimate goal for the final paint is to form a continuous film at a low temperature (2° C.). To achieve this, we first neat to find the MFFT of the neat latex material itself. If the neat latex material is above an MFFT of 2° C., we will add coalescent at different phr (% coalescent on latex solids) levels to allow the latex to reach 2° C. To reach that temperature, we can do a linear regression of the phr levels. This will allow us to determine an appropriate amount of coalescent to add to the final paint formulation.

### Test Procedure:

1. Turn water source, MFFT instrument, and nitrogen source on in that order
2. Let MFFT instrument equilibrate ˜15 minutes
3. Raise lid on the instrument and place the film caster (˜6 WFT) at the cold end (0°) of the bar
4. Our film caster is sectioned into individual squares allowing us to test up to five latex samples at a time
5. Add samples to film caster
6. Draw down samples from cold end to the warm end (18° C.) of the MFFT bar
7. Lower the lid on the instrument
8. Samples will be ready to evaluate in approximately 1-2 h
9. New MFFT bar instruments are equipped with a cursor. Moving the cursor to the MFFT point of a sample, the temperature value will be shown on a digital display

### Results

Twenty three acetal coalescents were made and are listed in Table 1, with synthesis details in Materials and Methods. Linear and branched aldehydes from C4 to C12 were used as starting materials, along with glycol ether solvents DE, DP, DB and MTG. Significantly, only 5 of the prototypes have existing CAS registry numbers as known compounds. These known compounds were previously studied only as surface-active agents (surfactants) or fragrance fixatives.

All of the e acyclic acetal materials passed the ASTM screening test for VOC as neat materials with values less than 7%, and typically less than 4% (Table 1), indicating that these materials when used as paint additives would not contribute in any significant way to the VOC content of the paint. In some cases, the observed VOC content can be attributed to residual starting materials in the preparation. A purification step may be added to improve product purity and reduce apparent VOC content. Odor is undesirable in a paint additive, and a low volatility material is often associated with low odor. In contrast to the acetal coalescents, the VOC content of Texanol™ is 100%.

TABLE 1
Volatility screening to estimate VOC content.

| Ex # | MW | VOC |
|---|---|---|
| Texanol ™ | 216.32 | 100% |
| 1 | 378.54 | 0.04 |
| 2 | 406.60 | 0.89 |
| 3 | 434.65 | 0.01 |
| 4 | 448.68 | 2.50 |
| 5 | 392.57 | 3.61 |
| 6 | 438.60 | 0.03 |
| 7 | 434.65 | 0.07 |
| 8 | 434.65 | 1.18 |
| 9 | 420.62 | 6.08 |
| 10 | 448.68 | 1.26 |
| 11 | 476.73 | 2.77 |
| 12 | 480.68 | 1.26 |
| 13 | 350.49 | 2.91 |
| 14 | 378.54 | 2.51 |
| 15 | 406.60 | 2.94 |
| 16 | 378.54 | 2.30 |
| 17 | 322.44 | 2.27 |
| 18 | 350.49 | 3.14 |
| 19 | 378.54 | 3.25 |
| 20 | 350.49 | 3.98 |
| 21 | 378.54 | 1.22 |
| 22 | 378.54 | 0.01 |
| 23 | 434.65 | 0.01 |

The most important performance feature of a coalescent is its ability to reduce the film-forming temperature of a latex paint. This property is evaluated by the MFFT test, with results shown in Table 2. The additive content required to form a visually uniform film at low temperatures is expressed relative to latex resin content (parts per hundred resin; phr) for three different resin types. The resins tested (Rhoplex^{TH} SG30, Acronal™ 296D and Encor™ 379) represent the main types of resins used globally; respectively acrylic, styrene acrylic and vinyl acrylic. For comparison, the phr of Texanol™ in the three resins is included in the top row.

The coalescing efficiency of Texanol™ was confirmed in this study, with good efficiencies in acrylic and styrene acrylic and vinyl acrylic latex resins. For the acetal coalescents, less than 8 phr was required to achieve a uniform film at 4.4° C., and typically less than 10 phr for film formation at 1.67° C.

TABLE 2
Minimum film forming temperature (MFFT) screen for latex
coalescing efficiency.

| | MFFT, phr for 4.4 and 1.67° C. | | | | | |
|---|---|---|---|---|---|---|
| | Rhoplex ™ SG30 | | Acronal ™ 296D | | Encor ™ 379 | |
| Ex# | 4.4° C. | 1.67° C. | 4.4° C. | 1.67° C. | 4.4° C. | 1.67° C. |
| | 4.01 | 5.26 | 6.30 | 7.77 | 3.10 | 4.94 |
| 1 | 5.15 | 6.81 | 5.61 | 6.83 | 2.77 | 4.52 |
| 2 | 4.26 | 5.59 | 6.25 | 7.53 | 2.81 | 4.58 |
| 3 | 5.47 | 7.17 | 5.69 | 6.89 | 3.49 | 5.73 |
| 4 | 4.57 | 5.99 | 5.80 | 7.05 | 2.50 | 4.11 |
| 5 | 4.32 | 5.68 | 5.51 | 6.71 | 2.49 | 4.08 |
| 6 | 6.46 | 8.55 | 7.40 | 8.99 | 3.47 | 5.71 |
| 7 | 7.38 | 9.57 | 6.45 | 7.86 | 4.49 | 7.23 |
| 8 | 7.71 | 10.10 | 7.01 | 8.52 | 4.81 | 8.11 |
| 9 | 7.72 | 10.06 | 7.32 | 8.93 | 4.55 | 7.49 |
| 10 | 7.03 | 9.11 | 6.65 | 8.13 | 4.99 | 8.32 |
| 11 | 7.51 | 9.77 | 7.24 | 8.70 | 7.09 | 11.63 |
| 12 | 5.90 | 7.76 | 7.51 | 9.17 | 3.15 | 5.19 |
| 13 | 4.56 | 5.95 | 5.47 | 6.66 | 2.43 | 4.00 |
| 14 | 4.43 | 5.80 | 5.06 | 6.16 | 2.34 | 3.83 |
| 15 | 4.28 | 5.64 | 5.17 | 6.29 | 2.54 | 4.12 |
| 16 | 4.50 | 5.91 | 5.23 | 6.36 | 2.35 | 3.86 |
| 17 | 5.32 | 7.03 | 6.57 | 8.04 | 2.84 | 4.64 |
| 18 | 4.10 | 5.35 | 5.07 | 6.17 | 2.17 | 3.56 |
| 19 | 3.79 | 5.00 | 5.04 | 6.14 | 2.22 | 3.63 |
| 20 | 3.98 | 5.25 | 5.19 | 6.30 | 2.28 | 3.73 |
| 21 | 4.05 | 5.31 | 4.99 | 6.08 | 2.20 | 3.60 |
| 22 | 5.68 | 7.53 | 5.77 | 7.01 | 2.72 | 4.49 |
| 23 | 7.28 | 9.52 | 6.98 | 8.47 | 4.58 | 7.47 |

The invention has been described in detail with reference to the embodiments disclosed herein, but it will be understood that variations and modifications can be effected within the spirit and scope of the invention.

We claim:

1. A composition comprising:
(1) a compound according to Formula I: 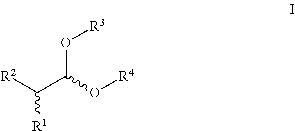
wherein:
R¹ is hydrogen, or (C₁₋₁₂)alkyl;
R² is (C₁₋₁₂)alkyl;
R³ is 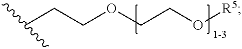
R⁴ is 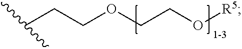
and
each R⁵ is (C₁₋₆)alkyl or (C₁₋₆)alkenyl; and
(2) a latex polymer;
wherein the compound of Formula I is present from about 1 to about 20 phr relative to the sum total of the latex polymer.

2. The composition of claim 1, wherein the latex polymer has a T_{g} in the range of from about −20° C. to about 100° C.

3. The composition of claim 1, wherein the latex polymer has a T_{g} in the range of from about 2° C. to about 60° C.

4. The composition of claim 1, wherein the compound of Formula I is present from about 1 to about 12 phr relative to the sum total of the latex polymer.

5. The composition of claim 1, wherein composition is characterized by having a minimum film forming temperature of 1.67° C. according to ASTM D2354-10e.

6. The composition of claim 1, wherein when R¹ is hydrogen, R² is not ethyl, hexyl or decyl, and wherein when R¹ is methyl, R² is not methyl.

7. The composition of claim 1, wherein the compound of Formula I has a volatile organic content of less than 50 wt % according to ASTM D6886.

8. The composition of claim 1, wherein the compound of Formula I has a volatile organic content of less than 10 wt % according to ASTM D6886.

9. The composition of claim 1, wherein the latex polymer is chosen from an acrylic, a vinyl acrylic, a styrene butadiene or a styrene acrylic latex polymer.

10. The composition of claim 1, wherein the compound of Formula I is chosen from:

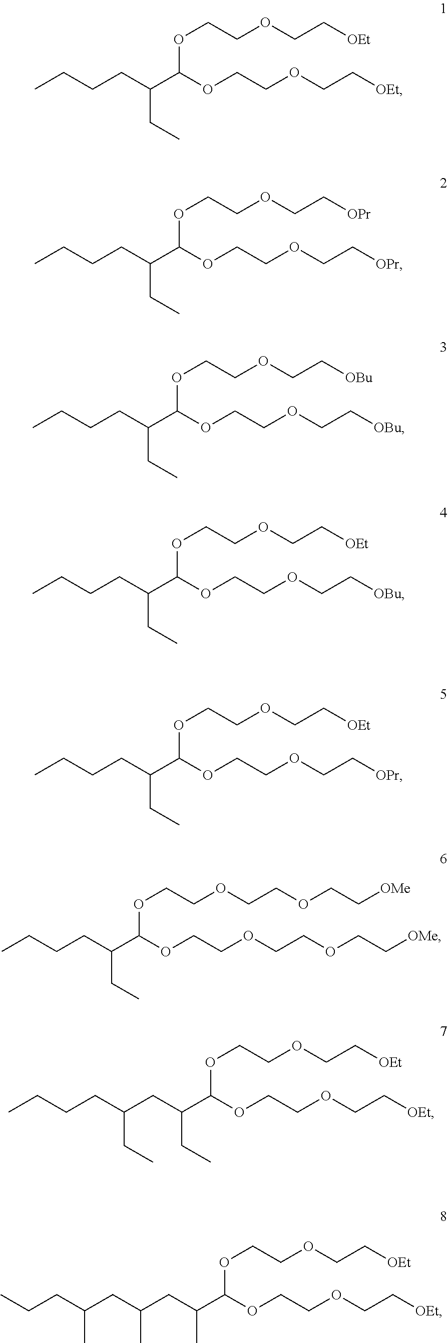

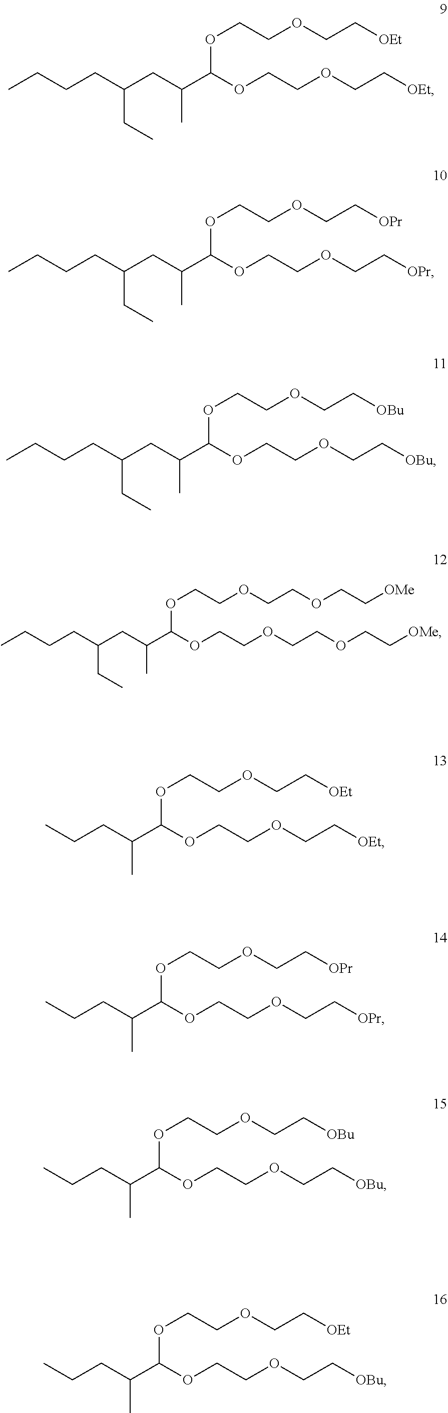

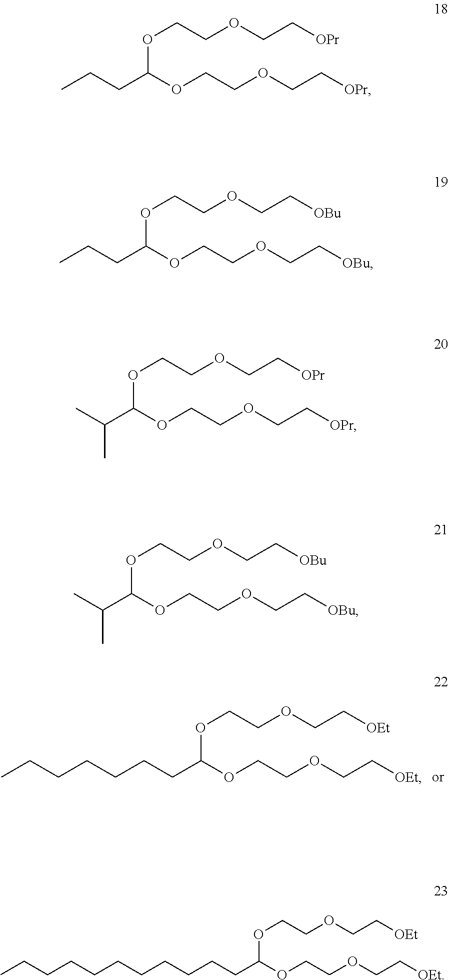

11. A compound according to Formula I: 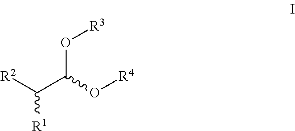
R¹ is hydrogen or (C₁₋₁₂)alkyl;
R² is (C₁₋₁₂)alkyl;
R³ is 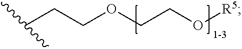
R⁴ is 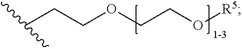
and
each R⁵ is (C₁₋₆)alkyl or (C₁₋₆)alkenyl,
wherein when R¹ is hydrogen, R² is not ethyl, hexyl or decyl,
wherein when R¹ is methyl, R² is not methyl.

12. The compound of claim 11, wherein R¹ is hydrogen; and R² is methyl, propyl, butyl, pentyl, heptyl, octyl, or nonyl.

13. The compound of claim 11, wherein R¹ is methyl; and R² is ethyl, propyl, butyl, pentyl, hexyl, heptyl, octyl, nonyl, or decyl.

14. The compound of claim 11, wherein R¹ is ethyl, propyl, butyl, pentyl, hexyl, heptyl, octyl, nonyl, or decyl.

15. The compound of claim 11, wherein R³ is

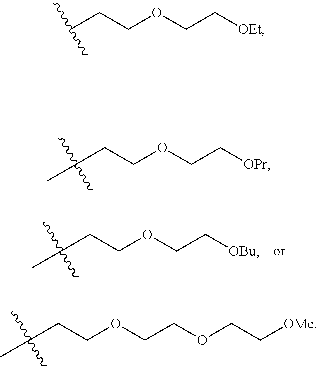

16. The compound of claim 15, wherein R⁴ is

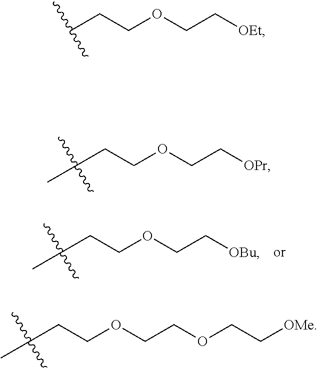

17. The composition of claim 1, wherein said compound is selected from the group consisting of structures 1-16:

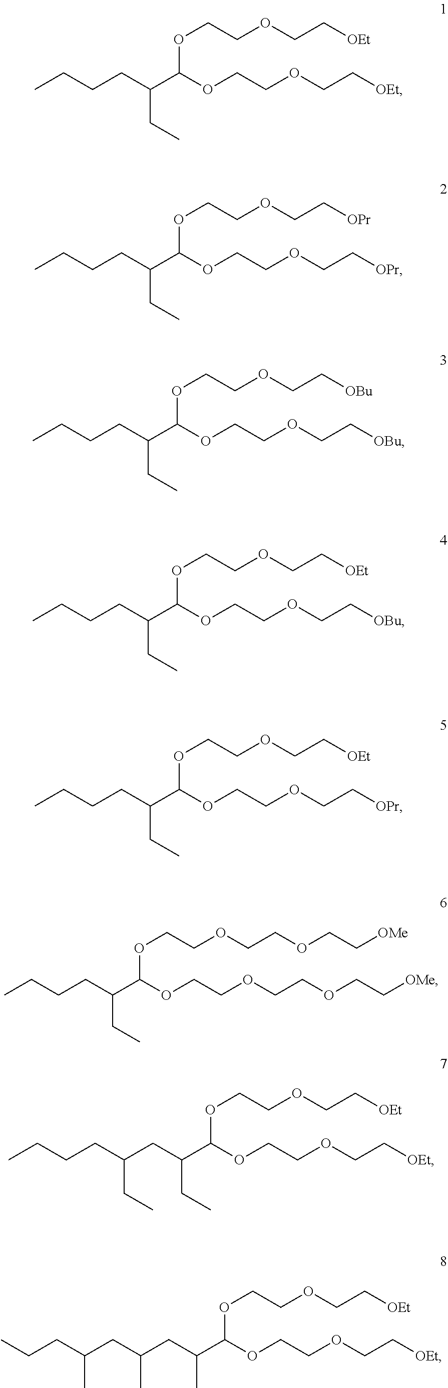

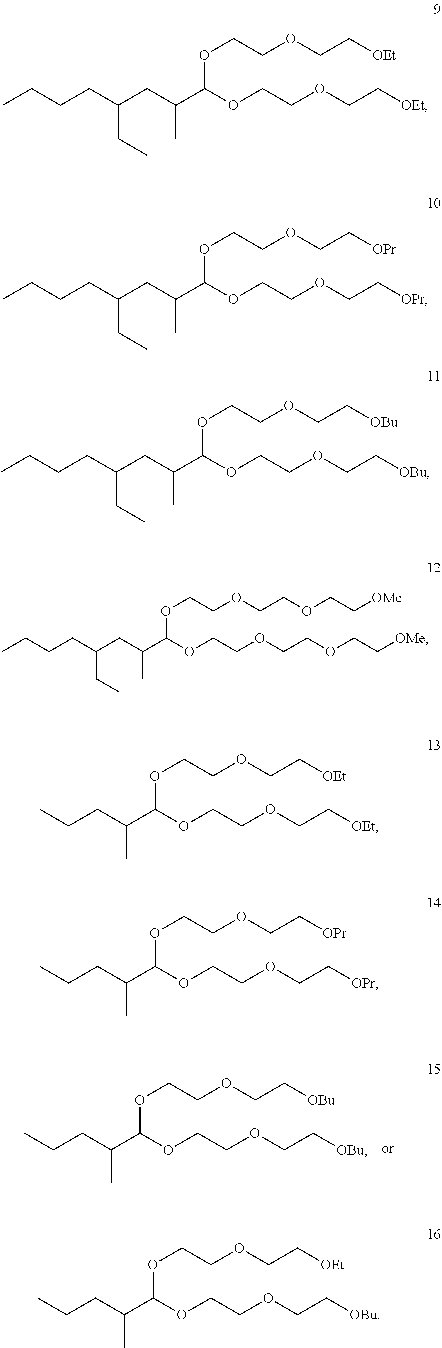

18. The compound of claim 10, wherein the compound has a volatile organic content of less than 50 wt % according to ASTM D6886.
